(19) United States
(12) Patent Application Publication
(10) Pub. No.: US 2023/0065895 A1
(43) Pub. Date: Mar. 2, 2023

(54) POXVIRAL-BASED VACCINE AGAINST SEVERE ACUTE RESPIRATORY SYNDROME CORONAVIRUS 2 AND METHODS USING THE SAME

(71) Applicant: ACADEMIA SINICA, Taipei City (TW)

(72) Inventors: Wen Chang, Taipei City (TW); Rakesh Kulkarni, Taipei City (TW); Shiu-Lok Hu, Redmond, WA (US)

(73) Assignee: ACADEMIA SINICA, Taipei City (TW)

(21) Appl. No.: 17/870,070

(22) Filed: Jul. 21, 2022

Related U.S. Application Data
(60) Provisional application No. 63/224,212, filed on Jul. 21, 2021.

(51) Int. Cl. A61K 39/215; A61K 39/39; A61K 9/00

(52) U.S. Cl. CPC A61K 39/215; A61K 39/39; A61K 9/0019; A61K 9/0014; A61K 2039/5256

(57) ABSTRACT

The present invention relates to a recombinant poxviral vector for use in vaccinating a subject against SARS-CoV-2. The present invention also provides vaccination regimens using the recombinant poxviral vector, which confers protective immunity against SARS-CoV-2.

### RELATED APPLICATIONS

This application claims the benefit of U.S. provisional application No. 63/224,212, filed Jul. 21, 2021 under 35 U.S.C. § 119, the entire content of which is incorporated herein by reference.

### REFERENCE. TO AN ELECTRONIC SEQUENCE LISTING

The contents of the electronic sequence listing (2022_07_21_ACA0143US_Sequence_Listing_5992-0366PUS2.xml; Size: 14,582 bytes; and Date of Creation: Jul. 18, 2022) is herein incorporated by reference in its entirety.

### TECHNOLOGY FIELD

The present invention relates to a recombinant poxviral vector for use in vaccinating a subject against SARS-CoV-2. The present invention also provides vaccination regimens using the recombinant poxviral vector, which confers protective immunity against SARS-CoV-2.

### BACKGROUND OF THE INVENTION

Severe acute respiratory syndrome coronavirus 2 (SARS-CoV-2), a member of the Betacoronavirus family, is causing a global pandemic and, as of April 2021, has infected more than 140 million people worldwide and resulted in 3 million deaths (https://covid19.who.int/) (1, 2). Compared to two other highly pathogenic coronaviruses, SARS-CoV (3) and Middle east respiratory syndrome coronavirus (MERS-CoV) (4), SARS-CoV-2 has proven more difficult to contain (5). Consequently, an effective vaccine to halt the spread of SARS-CoV-2 is urgently needed.

SARS-CoV-2 is an enveloped single-stranded positive-sense RNA virus, whose Spike protein (S) on the virion surface mediates virus entry into target cells (6-8). Spike protein has S1 and S2 components and, similar to other type 1 viral fusion proteins, the S1 subunit contains a receptor-binding domain (RBD) that binds to its host cell receptor, angiotensin converting enzyme 2 (ACE2) (9), whereas the S2 subunit mediates membrane fusion (10). The S protein of some SARS-CoV-2 strains requires cleavage by the cellular serine protease TMPRSS2 during cell entry (8, 11). Neutralizing antibodies from convalescent patients recognize S protein, making it a good vaccine target (12, 13). S protein is also a major target of T cell responses to SARS-CoV-2 (14, 15). Although several SARS-CoV-2 vaccines have been developed using mRNA technology (16-18) and adenovirus vectors (19-21), their efficacy in preventing virus spread among humans remains to be fully established. In addition, concerns have been raised of adverse effects following vaccination (22-24), implying that improvements to currently available SARS-CoV-2 vaccines are essential and will necessitate ongoing vaccine development.

### SUMMARY OF THE INVENTION

The present invention is based, at least in part, on the development of a recombinant poxviral vector for use in vaccinating a subject against SARS-CoV-2. The recombinant poxviral vector successfully confers protective immunity against SARS-CoV-2, with a single dose or prime-boost combinations, at least including induction of neutralizing antibodies and TH1-biased immune responses and effector memory CD8+ T cells responses against SARS-CoV-2, and reducing damages in organs or issues caused by SARS-CoV-2 infection.

Accordingly, in a first aspect, the present invention provides a recombinant poxviral vector which comprises a polynucleotide encoding a SARS-CoV-2 spike protein incorporated in a poxviral vector for use in vaccinating a subject against SARS-CoV-2.

In some embodiments, the poxviral vector is an orthopox viral vector.

In some embodiments, the orthopox viral vector is selected from the group consisting of a camelpox viral vector, a cowpox viral vector, a monkey pox viral vector, a smallpox viral vector and a vaccinia vial vector.

In some embodiments, the vaccinia vial vector is modified vaccine Ankara (MVA) or v-NY.

In some embodiments, the recombinant poxviral vector lacks a functional thymidine kinase gene.

In some embodiments, the polynucleotide is operatively-linked to a promoter.

In some embodiments, the promoter is a poxviral promoter e.g. a vaccinia viral early and late dual promoter.

In some embodiments, the SARS-CoV-2 spike protein comprises the amino acid sequence of SEQ ID NO: 1, 2, 3, 4 or 5, or a functional variant thereof.

In another aspect, the present invention provides an immunogenic composition against SARS-CoV-2 which comprises an effective amount of a recombinant poxviral vector as described herein and a physiologically acceptable vehicle.

In some embodiments, the immunogenic composition further comprises an adjuvant.

Also provided is use of a recombinant poxviral vector as described herein or an immunogenic composition thereof for manufacturing a medicament for vaccinating a subject against SARS-CoV-2.

The present invention further provides a method for vaccinating a subject against SARS-CoV-2, comprising administering to the subject an effective amount of a recombinant poxviral vector as described herein or an immunogenic composition thereof.

In some embodiments, the recombinant poxviral vector or the immunogenic composition is administered via a route selected from the group consisting of intramuscular injection, subcutaneous injection, intranasal administration, intradermal injection, skin scarification and oral administration and any combination thereof.

In some embodiments, the recombinant poxviral vector or the immunogenic composition is administered to the subject once or more than once.

In some embodiments, the method of the present invention comprises a first administration, followed by a second administration, of the recombinant poxviral vector or the immunogenic composition.

In some embodiments, the first administration and the second administration are intramuscular injection.

In some embodiments, the first administration is skin scarification and the second administration is intramuscular injection.

In some embodiments, the second administration is about four weeks after the first administration.

In some embodiments, the same dose is given in the first administration and the second administration.

In some embodiments, a higher dose is given in the first administration than in the second administration.

In some embodiments, the method of the present invention is effective in inducing neutralizing antibodies and T_{H}1-biased immune responses and effector memory CD8+ T cells specifically against SARS-CoV-2 in the subject.

In some embodiments, the method of the present invention is effective in reducing a disease or condition caused by SARS-CoV-2 infection in the subject.

In some embodiments, the disease or condition includes damages in organs or tissues in the subject, selected from the group consisting of lung, gastrointestinal tract, heart, kidney, liver, adrenal glands and/or testis.

In some embodiments, the disease or condition includes a pathological condition in lung, selected from the group consisting of diffuse congestion, shrinking of alveoli, hemorrhaging, immune cell infiltration and any combination thereof.

In some embodiments, the recombinant poxviral vector comprises v-NY-S (deposit accession number BCRC970077 or CNCM I-5857) and/or MVA-S.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The patent or application file contains at least one color drawing. Copies of this patent or patent application publication with color drawing will be provided by the USPTO upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A to 1D include charts showing generation and characterization of v-NY-S and MVA-S. (FIG. 1A). Schematic representation of the tk locus in the viral genomes of MVA-S and v-NY-S. The red box represents the ORF encoding SARS-CoV-2 S protein and the blue box represents the lacZ ORF. The small triangles represent viral promoters that drive gene transcription (FIG. 1B). Surface detection of SARS-CoV-2 S protein expressed from MVA-S and v-NY-S. BHK21 and BSC40 cells were infected with MVA-S (blue line) or v-NY-S (red line), respectively, harvested at 12 hours post-infection (hpi), stained with anti-RBD antibody, and then analyzed by flow cytometry. (FIG. 1C). Immunofluorescence staining of SARS-CoV-2 S protein in cells infected with MVA-S and v-NY-S. BHK21 and BSC40 cells were infected with MVA-S or v-NY-S at an MOI of 5 and fixed at 12 hpi with 4% paraformaldehyde, stained with anti-RBD antibody (green), and then photographed. Cell nuclei were stained with DAPI (blue). (FIG. 1D). Immunoblot of SARS-CoV-2 S protein expressed by MVA-S and v-NY-S. BHK21 cells were infected with MVA or MVA-S; BSC40 cells were infected with v-NY or v-NY-S, respectively, and harvested at 12 hpi for immunoblot analyses with anti-S2 antibody. Vaccinia D8 protein was used as a control.

FIGS. 2A to 2G include charts showing that prime-boost MVA/MVA, vNY1/MVA and vNY5/MVA vaccination regimens elicited SARS-CoV-2 S protein-specific neutralizing antibodies in C57BL/6 mice. (FIG. 2A). Summary and timeline of the three prime-boost vaccination regimens and analyses. (FIG. 2B). Primary and secondary sera from immunized mice recognized SARS-CoV-2 S protein on cell surfaces. Mouse sera collected 4 weeks after priming (1º sera) and 2 weeks after boosting (2º sera) were assessed for SARS-CoV-2-specific IgG antibodies by flow cytometry using SF9 cells infected with either S-BAC (red line) or WT-BAC (black line). (FIG. 2C). Quantification of anti-spike antibody titers in 1º and 2º sera from mice (as shown in FIG. 2B) using the mean fluorescence intensity (MFI) value from FACS. Numbers of mice for 1º and 2º sera collection are identical: PBS vs. PBS/PBS control (n=3); MVA vs. MVA/MVA (n=5); vNY1 vs. vNY1/MVA (n=5), and vNY5 vs. v-NY5/MVA (n=5). Data are represented as mean±SD. (FIG. 2D). Immunoblot analyses of recombinant SARS-CoV-2 S protein using 1º and 2º sera (1:100) from immunized mice. (FIG. 2E). Neutralization assays of 2º sera collected from vaccinated mice using (i) Pseudovirus and (ii) SARS-CoV-2 virus infection: PBS/PBS control (n=3); MVA/MVA (n=5); vNY1/MVA (n=5); and vNY5/MVA (n=5). The dotted line represents assay limits of detection. (FIG. 2F). Quantification of anti-spike antibodies in mouse sera collected at 0.5 and 4.5 months after vaccination regimens using SF9 cells infected with WT-BAC or S-BAC. (FIG. 2G). Pseudovirus neutralization assay using mouse sera collected at 0.5 months and 4.5 months after vaccination regimens: PBS/PBS control (n=3); MVA/MVA (n=5); and vNY1/MVA (n=5). ns—not significant. The dotted line represents assay limits of detection.

FIGS. 3A to 3E include charts showing that MVA/MVA, vNY1/MVA and vNY5/MVA vaccination regimens induce Till-biased immune responses. (FIG. 3A). End-point titers of SARS-CoV-2 spike-specific IgG2C and IgG1 antibodies in mouse sera collected 2 weeks after vaccination regimens. (FIG. 3B). End-point titer IgG2C/IgG1 ratio calculated based on data from (A) (n=5 for each group). (FIG. 3C). ELISpot analyses of mouse splenocytes collected 4 weeks after vaccination regimens for their expression of IL-2, IFN-γ, TNF-γ, IL4 and IL6 cytokines (n=5 for each group). Data represented as mean±SEM. SFC—spot-forming cells. (FIG. 3D & FIG. 3E). SARS-CoV-2 spike-specific CD8⁺ (in FIG. 3D) and CD4⁺ (in FIG. 3E) T effector memory cells (CD44⁺CD62L⁻) in splenocytes, as detected by flow cytometry (n=5 for each group). Data represented as mean±SD. ns—not significant.

FIGS. 4A to 4E include charts showing that MVA/MVA, vNY1/MVA and vNY5/MVA prime-boost vaccination regimens generated SARS-CoV-2 spike-specific neutralizing antibodies in Syrian hamsters. (FIG. 4A). Timeline for hamster immunization and sera collection. (FIG. 4B). Primary and secondary sera from immunized hamsters recognized SARS-CoV-2 S protein on cell surfaces. Hamster sera collected 4 weeks after priming (1º sera) and 2 weeks after boosting (2º sera) were assessed for SARS-CoV-2-specific IgG antibodies by flow cytometry using SF9 cells infected with either S-BAC (red line) or WT-BAC (black line). (FIG. 4C). Quantification of anti-spike antibody titers in 1º and 2º sera from hamsters in B, using the mean fluorescence intensity (MFI) value from FACS. Numbers of hamsters for 1º and 2º sera collection are identical: PBS vs. PBS/PBS control (n=15); MVA vs. MVA/MVA (n=10); vNY1 vs. vNY1/MVA (n=10), and vNY5 vs. v-NY5/MVA (n=10). Data are represented as mean±SD. (FIG. 4D). Immunoblots of 1º and 2º sera (1:20) from immunized hamsters using recombinant SARS-CoV-2 S protein. (FIG. 4E). Neutralization assays of 2º sera collected from vaccinated hamsters using (i) pseudovirus and (ii) SARS-CoV-2 virus infection: PBS control (n=12); MVA/MVA (n=10); vNY1/MVA (n=10); and vNY5/MVA (n=10). The dotted line represents assay limits of detection.

FIGS. 5A to 5E include charts showing that hamsters subjected to the MVA/MVA, vNY1/MVA or v-NY5/MVA vaccination regimens were protected against intranasally-administered SARS-CoV-2 infection. (FIG. 5A). Timeline of the immunization and challenge experiments. Hamsters immunized with one of three prime-boost vaccination regimens (MVA/MVA, vNY1/MVA or vNY5/MVA), or placebo (PBS) as a control, were challenged i.n. with 1×10⁵ PFU SARS-CoV-2 virus, before harvesting lungs at 3 or 7 d.p.i. (FIG. 5B). Weight change of hamsters within 3 days of SARS-CoV-2 challenge. Data represented as mean±SEM. (FIG. 5C). TCID₅₀value of SARS-CoV-2 in lung tissue of hamsters at 3 d.p.i. after SARS-CoV-2 challenge: PBS/PBS control (n=12); MVA/MVA (n=5); vNY1/MVA (n=10); and vNY5/MVA (n=10). (FIG. 5D). Weight change of hamsters within 7 days of SARS-CoV-2 challenge: PBS/PBS control (n=3); and MVA/MVA (n=5). Data represented as mean±SEM. (FIG. 5E). SARS-CoV-2 genomic RNA in lungs of MVA/MVA-immunized hamsters at 7 d.p.i. after SARS-CoV-2 challenge: PBS/PBS control (n=3); and MVA/MVA (n=5). Unless stated otherwise, data are represented as mean±SD. The dotted line represents assay limits of detection.

FIGS. 6A to 6F include charts showing lung pathology and immunohistochemistry of hamsters after SARS-CoV-2 challenge. (FIG. 6A). H&E and immunohistochemical staining of lungs of the placebo (PBS/PBS) infection hamster group at 3 d.p.i. H&E staining showed severe bronchointerstitial pneumonia with the alveolar walls thickened by edema, capillary congestion and variable immune cell infiltration. Immunohistochemistry of SARS-CoV-2 NP protein revealed prominent peribronchiolar staining, with the vascular endothelia frequently disrupted by immune infiltrates. (FIG. 6B, FIG. 6C & FIG. 6D). H&E and immunohistochemical staining of lungs from the vNY1/MVA (FIG. 6B), vNY5/MVA (FIG. 6C) and MVA/MVA (FIG. 6D) hamster groups at 3 d.p.i. Compared to the placebo (PBS/PBS) infection hamster group, lung architecture was better preserved, there was much less immune cell infiltration, and SARS-CoV-2 NP staining signal was barely detectable. (FIG. 6E). H&E and immunohistochemical staining of lungs of the placebo (PBS/PBS) infection hamster group at 7 d.p.i. H&E staining revealed prominent type II pneumocytic hyperplasia with variable immune cell infiltration. Immunohistochemistry of SARS-CoV-2 NP protein detected dispersed positive signals at the edges of regenerative foci. (FIG. 6F). MVA/MVA-immunized hamsters displayed minimal lung pathology and scant SARS-CoV NP immunolabeling at 7 d.p.i. The enlarged views of H&E and immunohistochemistry-stained regions are marked by red boxes. The scale bar represents 50 μm.

FIGS. 7A to 7F include charts showing that single-dose vNY1 or vNY5 vaccination partially protected hamsters from intranasally-administered SARS-CoV-2 infection. (FIG. 7A). Timeline showing the immunization and challenge experiment. Hamsters immunized with a single dose of vNY1, vNY5 or placebo (PBS) were challenged i.n. with 1×10⁵ PFU SARS-CoV-2 virus and then lungs were harvested at 3 d.p.i. (FIG. 7B). Pseudovirus neutralization assays of the 1º sera collected 2 weeks after vaccine priming in hamsters. PBS control (n=4); vNY1 (n=5); and vNY5 (n=5). Data represented as mean±SD. The dotted line represents assay limits of detection. (FIG. 7C). TCID₅₀ values for lungs from hamsters of the placebo (PBS), vNY1 and vNY5 groups at 3 d.p.i. after SARS-CoV-2 challenge (n=5 for each group). Data represented as mean±SD. The dotted line represents assay limits of detection. (FIG. 7D). H&E and immunohistochemical staining of lungs of the placebo (PBS) infection hamster group at 3 d.p.i. H&E staining revealed an identical pathology to that shown in FIG. 6A, revealing severe bronchointerstitial pneumonia with the alveolar walls thickened by edema, capillary congestion and variable immune cell infiltration. Immunohistochemistry of SARS-CoV-2 NP protein revealed prominent peribronchiolar staining, with the vascular endothelia frequently being disrupted by immune infiltrates. (FIG. 7E and FIG. 7F). H&E and immunohistochemical staining of hamster lungs primed with vNY1 (in FIG. 7E) or vNY5 (in FIG. 7F) at 3 d.p.i. The lung architecture was largely preserved, displaying reduced immune cell infiltration relative to the placebo infection group and SARS-CoV-2 NP protein was barely detectable by immunohistochemistry.

FIG. 8 shows weight change in C57BL/6 mice after immunization with one of three regimens.

FIGS. 9A to 9B include charts showing skin scarification in animals and wright change. (FIG. 9A). Images of skin scarification in Syrian hamsters at days 5,10 and 15 after primary immunization. (FIG. 9B) Weight change in Syrian hamsters after immunization with one of the three regimens.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20 or 10 amino acid residues).

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. As used herein, a “coding sequence” or a sequence “encoding” an expression product, such as a RNA or polypeptide, is a nucleotide sequence that, when expressed, results in the production of that RNA or polypeptide i.e., the nucleotide sequence encodes an amino acid sequence for that polypeptide. A coding sequence for a protein may include a start codon (usually ATG) and a stop codon. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

In general, a functional variant of a polypeptide is substantially identical to the reference sequence e.g. amino acid sequence identity of more than 80%, particularly about 85%-95% or more, such as at least about 95%, 96%, 97%, 98%, 99% or more, when the two sequences are aligned. To determine the percent identity of two sequences, the sequences can be aligned for optimal comparison purpose. In calculating percent identity, typically exact matches are counted. The determination of percent homology or identity between two sequences can be accomplished using a mathematical algorithm known in the art, such as BLAST and Gapped BLAST programs, the NBLAST and XBLAST programs, or the ALIGN program.

The SARS-CoV-2 spike protein is a characteristic structural component of the SARS-CoV-2 virion membrane that forms large protruding spikes on the surface of the virus. It contains S1 and S2 subunits where the S1 subunit contains a receptor-binding domain (RBD) that binds to angiotensin converting enzyme 2 (ACE2) as the host cell receptor of SARS-CoV-2, and the S2 subunit mediates membrane fusion. In one embodiment, a SARS-CoV-2 spike protein as described herein includes the SARS-CoV-2 spike protein from Wuhan-Hu-1 variant (SEQ ID NO: 1; YP009724390.1). In other embodiments, a SARS-CoV-2 spike protein as described herein include those from certain variants, including but not limited to, Alpha variant (B.1.1.7) (SEQ ID NO: 2; QWB50088.1), Beta variant (B.1.351) (SEQ ID NO: 3; QWA53303.1), Gamma variant (P.1) (SEQ ID NO: 4; QWB58007.1), and Delta variant (B.1.617.2) (SEQ ID NO: 5; QWB15066.1).

The term “recombinant” is used to describe a polynucleotide or nucleic acid having sequences that are not naturally joined together. A recombinant nucleic acid may be present in the form of a construct. The term “construct” as used herein may contain a given nucleotide sequence of interest, and some sequence required for expression of the nucleotide sequence of interest, such as a regulatory sequence. Constructs may be used for expressing the given nucleotide sequence or maintaining the given nucleotide sequence for replicating it, manipulating it or transferring it between different locations (e.g., between different organisms). Constructs can be introduced into a suitable host cell for the above mentioned purposes. Typically, in a construct, the given nucleotide sequence is operatively linked to the regulatory sequence such that when the constructs are introduced into a host cell, the given nucleotide sequence can be expressed in the host cell under the control of the regulatory sequence. The regulatory sequence may comprises, for example and without limitation, a promoter sequence, a start codon, a replication origin, enhancers, an operator sequence, a secretion signal sequence and other control sequence (e.g., termination sequences). Preferably, constructs may further contain a marker sequence (e.g., an antibiotic resistant marker sequence) for the subsequent screening procedure.

A “viral vector” is a nucleic acid molecule which comprises viral sequences which can be packaged into viral particles and is capable of introducing a foreign nucleic acid into a cell of an individual. A “recombinant viral vector” as used herein refers to a recombinant viral construct comprising a virus genome and a heterologous polynucleotide, for example, encoding a foreign protein. The term “recombinant” may include any modification, alteration or engineering of a polynucleotide or protein in its native form or structure. The modification, alteration or engineering of a polynucleotide or protein may include, but is not limited to, deletion of one or more nucleotides or amino acids, deletion of an entire gene, codon-optimization of a gene, conservative substitution of amino acids and insertion of one or more heterologous polynucleotides. As used herein, the term “poxviral vector” refers to a viral vector from a virus member of the family poxviridae. The family poxviridae is characterized by a genome of double-stranded DNA. Preferably, the poxviral vector belongs to the orthopox and is selected from the group consisting of a camelpox viral vector, a cowpox viral vector, a monkey pox viral vector, a smallpox viral vector and a vaccinia vial vector. Vaccinia virus has been deployed successfully to eradicate smallpox worldwide (25, 26). One example of a vaccinia viral vector is modified vaccinia Ankara (MVA). The MVA strain is growth-restricted in mammalian cells and preclinical and clinical trials have demonstrated it to be quite a safe vaccine vector against viral diseases such as HIV, MERS-CoV and SARS-CoV (27-30). Alternatively, v-NY strain is replication-competent virus derived from the New York City Board of Health viral strain smallpox vaccine that displays reduced virulence compared to the standard smallox vaccine (Dryvax®). The v-NY strain has been described as a vector for the construction of recombinant vaccinia viruses, for example in Australia Patent No. AU608205B2, the relevant disclosures of which are incorporated by reference herein for the purposes or subject matter referenced herein.

As used herein, the term “immunogenic composition” refers to a composition capable of inducing an immune response, such as an antibody or cellular immune response, when administered to a subject. Persons skilled in the art can determine immunogenic responses by routine assays with respect to the determination of an immunogenic activity of antigens or immunogens of interest. Preferably, the immunogenic composition is formulated as a vaccine which can prevent, ameliorate, palliate or eliminate diseases/infections from the subject.

The present invention is based, at least in part, on the development of a recombinant poxviral vector which comprises a polynucleotide encoding a SARS-CoV-2 spike protein for use in vaccinating a subject against SARS-CoV-2. It is surprisingly found that such recombinant poxviral vector confers protective immunity against SARS-CoV-2. In particular, the recombinant poxviral vector as described herein comprises a poxviral genomic sequence with a promoter and a heterologous polynucleotide operably linked to the promoter encoding a SARS-CoV-2 spike protein.

The recombinant poxviral vector according to the present invention can be prepared by any technique known to those of ordinary skill in the art. In particular, they can be prepared by homologous recombination between a poxvirus and a plasmid carrying, inter alia, polynucleotides encoding a SARS-CoV-2 spike protein. The homologous recombination occurs after infection of the said virus and transfection of the plasmid into an appropriate cell line.

In certain embodiments, the recombinant poxviral vector as described herein is a recombinant MVA vector encoding a SARS-CoV-2 spike protein, MVA-S.

In certain embodiments, the recombinant poxviral vector as described herein is a recombinant v-NY vector encoding a SARS-CoV-2 spike protein, v-NY-S (deposited on Jul. 14, 2021 with the Food Industry Research and Development Institute (FIRDI), Hsinchu city, Taiwan, accession number: BCRC970077; and deposited on Jul. 13, 2022 with the Collection nationale de cultures de micro-organismes (CNCM), Paris, France, France, accession number: CNCM 1-5857).

For administration, an effective amount of the recombinant poxviral vector may be formulated with a physiologically acceptable carrier into a composition of an appropriate form for the purpose of delivery and absorption. Preferably, an effective amount of the recombinant poxviral vector is formulated as an immunogenic composition which induces one or more immune responses against SARS-CoV-2. As used herein, “physiologically acceptable” means that the carrier is compatible with the active ingredient in the composition, and preferably can stabilize said active ingredient and is safe to the receiving individual. Such physiologically acceptable carriers are well known in the art. In some embodiments, the purified viral vector is formulated and administered as a sterile solution while it is also possible to utilize lyophilized preparations. Sterile solutions can be lyophilized or filled into pharmaceutical dosage containers. The pH of the solution generally is in the range of pH 3.0 to 9.5, e.g pH 5.0 to 7.5. The viral vector typically is in a solution having a suitable pharmaceutically acceptable buffer. In certain embodiments, the viral vector may be formulated into an injectable preparation. These formulations contain effective amounts of a viral vector, are either sterile liquid solutions, liquid suspensions or lyophilized versions and optionally contain stabilizers or excipients. A viral vector vaccine can also be aerosolized for intranasal administration.

The term “effective amount” used herein refers to the amount of an active ingredient to confer a desired biological effect in a treated subject or cell. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience. In some embodiments, an effective amount as used herein can be an amount effective in inducing protective immunity against SARS-CoV-2, such as induction of humoral and/or neutralizing antibodies and/or T_{H}1-biased immune responses and effector memory CD8+ T cells against SARS-CoV-2, and reducing damages in organs or issues caused by SARS-CoV-2 infection. Specifically, a recombinant poxviral vector or a composition thereof as described herein can be administered to a subject or infected or transfected into cells in an amount of about at least about 10⁵ pfu to about 10⁹ pfu, for instance about 10⁶ pfu to about 10⁸ pfu, per dose, for example, about 10⁷ pfu per dose.

In certain embodiments, a composition comprising the viral vector may further comprise one or more adjuvants. The terms “adjuvant” and “immune stimulant” are used interchangeably and are defined as one or more substances that cause stimulation of the immune system. In certain embodiments, the compositions of the invention comprise aluminium as an adjuvant, e.g. in the form of aluminium hydroxide, aluminium phosphate, aluminium potassium phosphate, or combinations thereof. One example is an aluminum hydroxide wet gel suspension such as 2% Alhydrogel (Invitrogen Inc.).

Administration of a recombinant poxviral vector and a composition thereof can be performed using standard routes of administration. Exemplified administration includes intramuscular injection, subcutaneous injection, intranasal administration, intradermal injection, skin scarification and oral administration.

It is for example possible to administer to a subject a recombinant poxviral vector according to the invention as single dose, or as a prime (first administration) and boosting (second administration). The period of time between prime and boost is generally one (1) week, two (2) weeks, four (4) weeks, six (6) weeks or eight (8) weeks, preferably 4 weeks or 8 weeks. It is also possible to perform more than one boosts where the subsequent boost is administered 2 weeks, 4 weeks, 6 weeks, 8 weeks or 12 weeks after the preceding boost. Preferably, the interval between two boosts is 4 weeks 8 weeks or 12 weeks.

In some embodiments, the recombinant poxviral vector or the immunogenic composition is administered to the subject once or more than once, such as twice, three times, four times, five times, six times or more.

In some embodiments, the method of the present invention comprises a first (prime) administration, followed by a second (boost) administration, of the recombinant poxviral vector or the immunogenic composition. The boost administration may be followed by additional boost administration as needed.

In some embodiments, the first administration and the second administration are intramuscular injection.

In some embodiments, the first administration is skin scarification and the second administration is intramuscular injection.

In some embodiments, the second administration is about four weeks after the first administration.

In some embodiments, the same dose is given in the first administration and the second administration.

In some embodiments, a higher dose is given in the first administration than that given in the second administration. For example, the dose in the first administration is about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold or 10-fold of the dose of the second administration.

In some embodiments, the recombinant poxviral vector given in the first administration and that given in the second administration are the same or different.

In some embodiments, the recombinant poxviral vector as used comprises MVA-S and/or v-NY-S.

In some embodiments, an effective amount of v-NY-S is administered first and an effective amount of MVA-S is administered later. In certain examples, the dose of v-NY-S in the prime administration is higher than the dose of MVA-S in the boost administration. For example, the dose of v-NY-S is about 5-fold of the dose of MVA-S. In some embodiments, v-NY-S is given in the first administration via skin scarification and MVA-S is given in the second administration via intramuscular injection.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, particularly SARS-CoV-2 infection.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

Vaccinia virus has been deployed successfully to eradicate smallpox worldwide (25, 26). Here, we generated SARS-CoV-2 vaccines using the MVA strain, as well as a v-NY strain previously employed as a vector for the first recombinant vaccinia virus (HIVAC-1e) used in FDA-approved clinical trials (44-48), both of which we engineered to express SARS-CoV-2 S protein. Unlike MVA, the v-NY strain is a replication-competent virus derived from the New York City Board of Health viral strain of smallpox vaccine (44-47) that displays reduced virulence compared to the standard smallpox vaccine (Dryvax®). Due to the different features of these two vaccinia virus strains, we tested different prime-boost combinations of both vaccines to establish an effective regimen for immunoactivation in C57BL/6 mice. We eventually chose two prime-boost regimens. The first regimen is to prime 5×10⁷ PFU of MVA-S virus intramuscularly (i.m.) in hamsters, wait for four weeks and boost i.m. these primed animals with 10⁷ PFU of MVA-S virus. The second regimen is to use skin scarification to introduce 10⁷ PFU (or 5×10⁷ PFU) v-NY-S into hamsters, wait for four weeks and then i.m. boost these primed animals with 10⁷ PFU of MVA-S virus. Two weeks after the boost, these vaccinated hamsters were challenged with SARS-CoV-2 virus using 1×10⁵ TCID₅₀ dosage and body weight of the animals was measured and the SARS-CoV-2 virus titers in lungs were determined at day 3 post infection (p.i.). Furthermore, we demonstrate that our vaccination regimens generated antibody and T cell responses in mice and protected Syrian hamsters from SARS-CoV-2 infection.

1. Material and Methods

1.1 Chemicals, Cells, Viruses and Animals

The following antibodies were used: SARS-CoV-2 anti-RBD antibody (40592-T62, Sino Biological); SARS-CoV-2 anti-spike S2 mouse mAb (GTX632604,GeneTex); rabbit monoclonal anti-SARS-CoV/SARS-CoV-2 nucleocapsid (NP) antibody (40143-R001,Sino Biological); FITC-conjugated goat anti-Rabbit IgG Ab (F1262, Sigma); HRP goat anti-mouse IgG Ab (31430, Pierce Biotechnology); HRP goat anti-hamster IgG Ab (PA1-28823, Invitrogen); Cy5-Goat anti-mouse IgG Ab (115-175-146, Jackson ImmunoResearch); FITC-Goat anti-hamster IgG Ab (11-4211-85, eBioscience); HRP-Conjugated IgG2C (PAI-29288, Invitrogen); HRP-conjugated IgG1 (PAI-74421, Invitrogen) and anti-CD3-PE/Cyanine7, (#100220, BioLegend), anti-CD4-FITC (#100510, BioLegend), anti-CD8-Pacific blue (#100725, BioLegend), anti-CD44-PE (#103008, BioLegend) and anti-CD62L-APC (#104412, BioLegend).

BSC40 cells were cultured in DMEM supplemented with 10% fetal bovine serum (FBS) (Gibco) and 1% penicillin-streptomycin (PS) (Gibco). BHK21 cells were cultured in RPMI medium supplemented with 10% FBS and 1% PS. HuTK-143 cells were cultured in MEM medium supplemented with 10% FBS and 1% PS. The v-NY strain of vaccinia virus was grown on BSC40 or HuTK-143 cells as described previously (69-74). The MVA strain of vaccinia virus (VR-1508, ATCC) was grown on BHK21 cells. SARS-CoV-2 TCDC #4 (hCoV-19/Taiwan/4/2020) is a local isolate and it was propagated on Vero-E6 cells. Eight-week-old female C57BL/6 mice (Charles River strain) were purchased from BioLASCO Taiwan Co. Ltd. Eight-week-old male and female Syrian hamsters (Mesocricetus auratus) were purchased from the National Laboratory Animal Center, Taiwan. All animal protocols were approved by the Institutional Animal Care and Utilization Committee of Academia Sinica and were conducted in strict accordance with the guidelines on animal use and care of the Taiwan National Research Council's Guide.

1.2 Construction of Recombinant Vaccinia Viruses

To generate recombinant vaccinia viruses expressing SARS-CoV-2 S protein (NCBI reference sequence NC_045512), a human codon-optimized open reading frame (ORF) encoding full-length SARS-CoV-2 S protein was inserted into a pSC11 plasmid and under regulatory control by an early and late p7.5k promoter to obtain pSC11-S plasmid (75). The pSC11-S plasmid was transfected into HuTK-143 cells infected with the wild type v-NY virus strain. Lysates were then harvested for multiple rounds of plaque purification of the recombinant virus, named v-NY-S, on HuTK-143 in the presence of 25 μg/ml 5-Bromo-2′-Deoxyuridine (BrdU), as described previously (73). The recombinant MVA strain expressing SARS-CoV-2 S protein, MVA-S, was generated as described for v-NY-S except that BHK21 cells were used and plaque purification was performed in the presence of X-gal (150 μg/ml). Both MVA-S and v-NY-S were subsequently amplified in roller bottles, and the virus stocks were partially purified using a 36% sucrose gradient and titrated prior to use, as described previously (76).

1.3 Immunofluorescence Staining of Cell Surface S Protein

BHK21 and BSC40 cells were infected respectively with MVA-S or v-NY-S at a multiplicity of infection (MOI) of 5 PFU/cell for 1 h, washed with PBS, and then incubated in growth media for a further 12 h. The cells were then washed with PBS and fixed with 4% paraformaldehyde, before being immunostained with SARS-CoV-2 anti-RBD antibody (40592-T62) at a dilution of 1:500 for 1 h at room temperature. Then, the cells were washed with PBS and stained for the secondary antibody FITC-conjugated goat anti-Rabbit IgG Ab (F1262, 1:500 dilution) for 1 h at room temperature, followed by staining with DAPI (5 μg/ml, D21490, Molecular Probes) for 5 min and mounting with Vectashield mounting solution (H-1000, Vector Laboratories). Images were taken using a Zeiss LSM 710 confocal microscope with a 63× objective lens, as described previously (77).

1.4 Prime-Boost Immunization Regimens in Mice and Hamsters

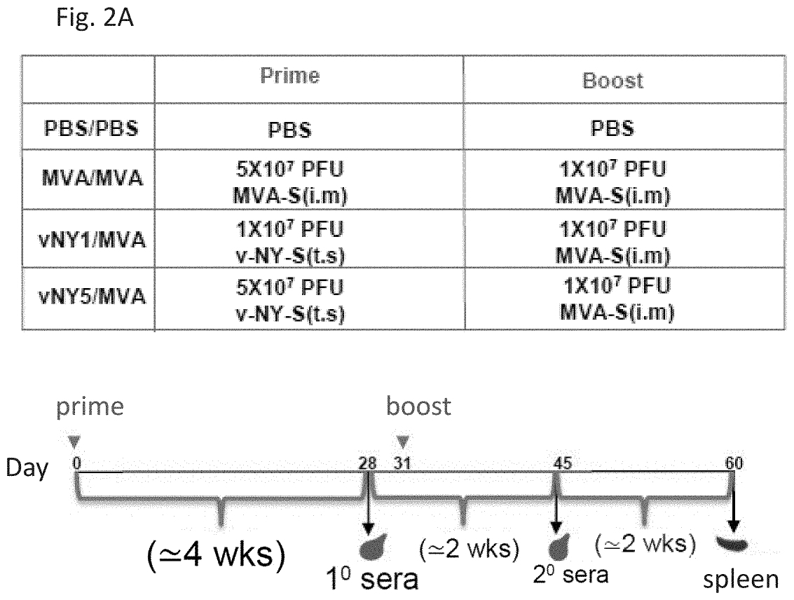

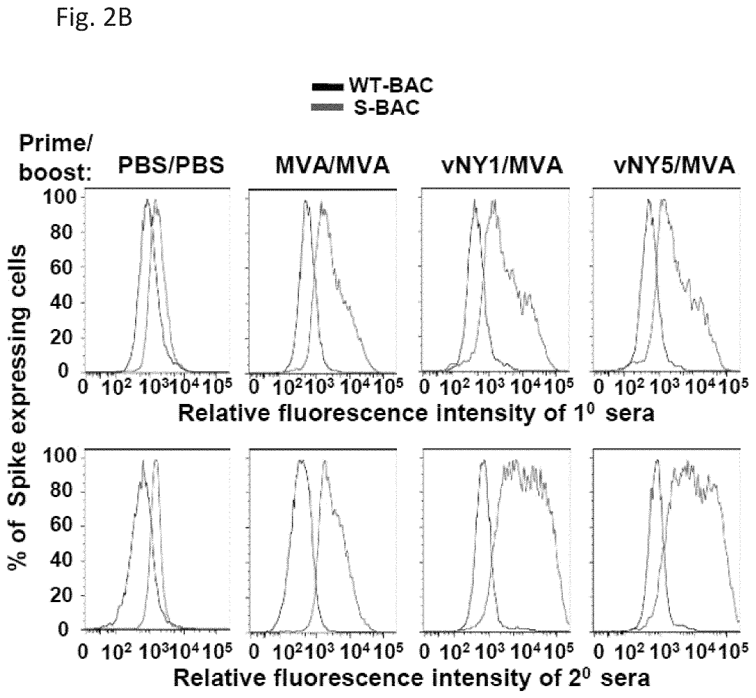

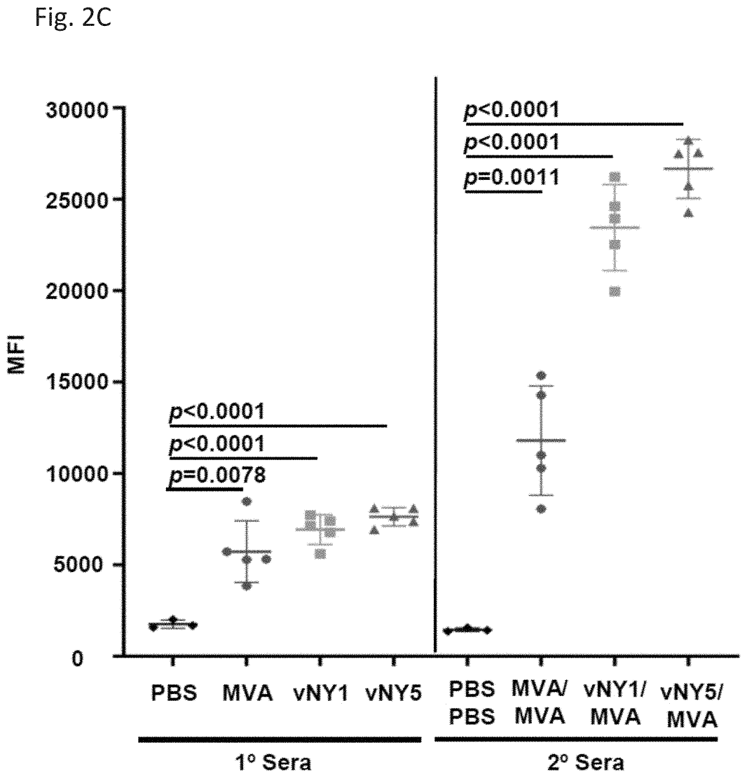

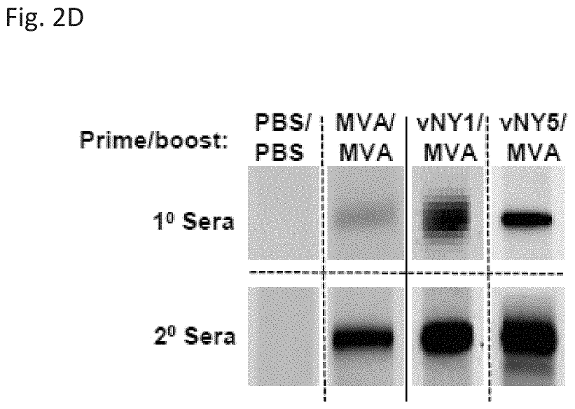

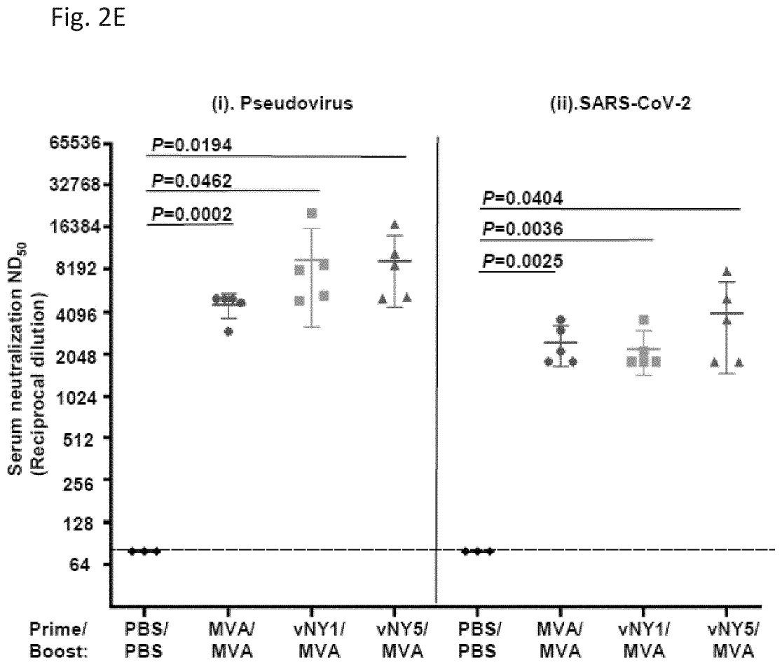

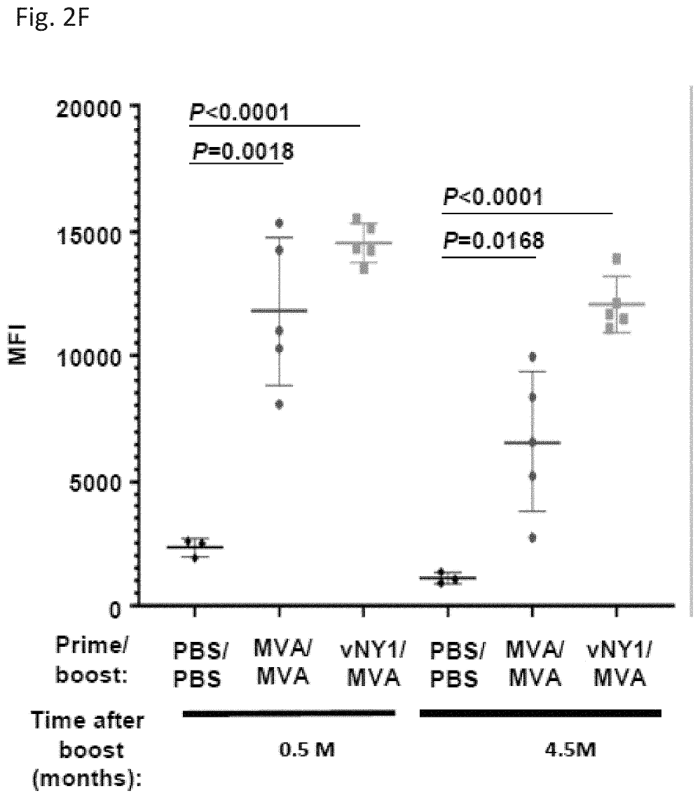

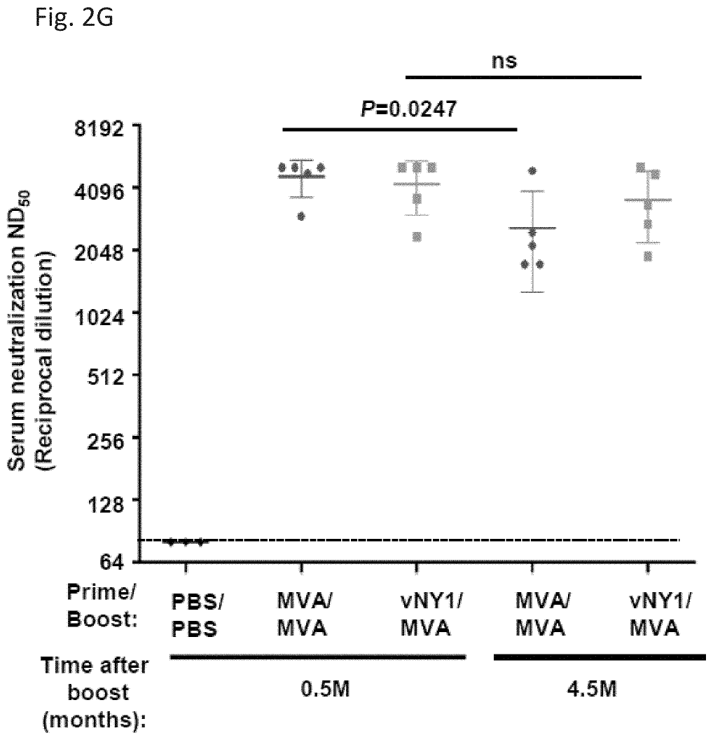

Eight-week-old female C57BL/6 mice and 8-week-old male and female Syrian hamsters were housed in the Animal Facility of Academia Sinica (Taipei, Taiwan) for at least 3 days prior to vaccination experiments. We used three regimens of two-dosage prime/boost immunization (depicted in FIG. 2A): (1) MVA/MVA—intramuscular (i.m.) inoculation of MVS-S in the right hind limb at 5×10⁷ PFU/animal, followed by an i.m. boost 4 weeks later of 1×10⁷ PFU/animal of MVA-S virus; (2) vNY1/MVA—tail scarification (t.$) of v-NY-S at 1×10⁷ PFU/animal, followed by an i.m. boost 4 weeks later of 1×10⁷ PFU/animal of MVA-S virus into the right hind limb; and (3) vNY5/MVA—t.s. of v-NY-S at 5×10⁷ PFU/animal, followed by an i.m. boost 4 weeks later of 1×10⁷ PFU/animal of MVA-S virus into right hind limb. As immunization controls, PBS buffer was used as a placebo vaccine for both priming and boosting shots. Blood was collected from immunized mouse cheeks and hamster gingival veins 4 weeks after priming and 2 weeks after boosting, as described previously (78, 79). Sera was prepared from blood and saved at −80° C. until use.

1.5 Immunoblotting

To measure SARS-CoV-2 S protein expression in cells infected with recombinant viruses, BSC40 and BHK21 cells (5×10⁵) were infected with v-NY-S and MVA-S, respectively, at an MOI of 5 PFU/cell and incubated for 12 h prior to cell harvesting. Cells were lysed with sample buffer and proteins were separated by sodium dodecyl sulfate-polyacrylamide gel electrophoresis (SDS-PAGE). Proteins were then transferred to nitrocellulose membranes (BioRad) using a wet transfer apparatus (Bio-Rad). The membranes were blocked in 5% non-fat milk solution at room temperature (r.t.) for 1 h and incubated overnight with SARS-CoV-2 spike S2 mouse mAb (GTX632604, 1:1000 dilution) at 4° C. The blots were then washed three times with PBST (PBS containing 0.1% Tween-20), incubated at r.t. with HRP goat anti-mouse IgG Ab (31430, 1:20,000) for 1 h and developed using a Western Lightening Enhanced Chemiluminescence kit (PerkinElmer) according to the manufacturer's protocol.

To test reactivity of immunized mouse and hamster sera to SARS-CoV-2 spike protein, the extracellular domain of spike protein from residue 14 to 1209, consisting of S1 and S2 but without the transmembrane domain, was expressed in HEK 293 cells and subsequently purified. The purified spike protein contained human complex type glycans, and exists as a trimer in solution with an apparent molecular weight between 170 to 235 kDa on SDS-PAGE (monomer), and ˜600 kDa (trimer) on Superose 6 size-exclusion chromatography. Purified spike protein (20 ng/well) was separated by SDS-PAGE, transferred to nitrocellulose membranes and blocked in 5% non-fat milk solution at r.t. as described above. The membrane was separated into multiple strips and each strip was incubated overnight with individual sera collected from immunized mice (1:100 dilution) or hamsters (1:50) at 4° C. These blots were then washed three times with PBST, incubated at r.t. with HRP goat anti-mouse (31430, 1:20,000) or HRP goat anti-hamster (PA1-28823, 1:5,000) antibodies for 1 h at r.t. and then developed using a Western Lightning Enhanced Chemiluminescence kit (PerkinElmer) according to the manufacturer's protocol.

1.6 Flow Cytometry Analysis of Cell-Surface SARS-CoV-2 S Protein Expression

To detect spike protein expression on the surface of cells infected with MVA-S or v-NY-S, BSC40 and BHK21 cells (5×10⁵) were infected with v-NY-S and MVA-S, respectively, at an MOI of 5 PFU/cell and incubated for 12 h, before being detached via treatment with 2 mM EDTA in PBS. Cells were incubated with SARS-CoV-2 anti-RBD antibody (40592-T62, 1:500) at 4° C. for 1 h. The cells were then washed with FACS buffer (PBS containing 2% FBS), stained with FITC-conjugated goat anti-Rabbit IgG Ab (F1262, 1:500) for 1 h at 4° C., washed with FACS buffer and analyzed by flow cytometry (BD LSR-II, BD Biosciences).

To detect anti-spike antibody in the sera of immunized mice and hamsters, SF9 insect cells were infected with either wild type baculovirus (WT-BAC) or a recombinant baculovirus (S-BAC) that expressed a chimeric SARS-CoV-2 S-gp64 protein in which the transmembrane and C-terminal regions of S protein were replaced by the transmembrane and C-terminal regions of baculovirus GP64 so that the S-gp64 fusion protein would be expressed on insect cell surfaces. These cells were cultured for 48 h before incubating with mouse (1:100 dilution) or hamster (1:20 dilution) serum in FACS buffer for 1 hour on ice. After two washes with FACS buffer, the cells were incubated with Cy5-Goat anti-mouse IgG Ab (115-175-146, 1:500) or FITC-Goat anti-hamster IgG Ab (11-4211-85, 1:100) for 30 min on ice, washed twice, resuspended in FACS buffer containing propidium iodide, and then analyzed by flow cytometry (BD LSR-II).

1.7 Pseudovirus Neutralization Assay

A lentiviral viral vector pseudotyped with SARS-CoV-2 S protein was generated and titered by the National RNA Technology Platform and Gene Manipulation Core, Academia Sinica, Taipei, Taiwan. Neutralization assays on pseudotyped virus were performed by the same core facility, as described previously (80) but with minor modifications. In brief, 1,000 units of the pseudotyped lentivirus with SARS-CoV-2 S protein were incubated at 37° C. for 1 h with serially-diluted sera obtained from vaccinated animals. The mixture was then added to HEK-293T cells expressing human ACE2 receptor (10⁴ cells/well of a 96-well plate) and incubated for 24 h at 37° C. This cell culture was then replaced with 100 μl of fresh DMEM plus 10% FBS, and the cells were incubated for another 48 h before undergoing luciferase assay. The reciprocal dilution of serum required for 50% inhibition of virus infection (ND₅₀) was assessed by measuring luciferase intensity.

1.8 SARS-CoV-2 Neutralization Assay

Serially diluted antibodies from immunized mice or hamsters were incubated at 37° C. for 1 h with 100 TCID₅₀ SARS-CoV-2 TCDC #4 (hCoV-19/Taiwan/4/2020). The mixtures were then added to pre-seeded Vero E6 cells for a 4-day incubation. Cells were fixed with 10% formaldehyde and stained with 0.5% crystal violet for 20 min. The plates were washed with tap water and scored for infection. The 50% protective titer was calculated according to the Reed & Muench Method (81).

1.9 Immunoglobulin ELISA for SARS-CoV-2 S-Specific Antibodies

Immunoglobulin ELISA was performed as described previously (17) with some modifications. Recombinant SARS-CoV-2 S protein (10 ng/well) was coated onto a 96-well plate (Costar assay plate, Corning, 3369) for 24 h at 4° C. The plates were then washed with PBST and blocked with 1% BSA in PBS solution for 1 h, followed by washes with PBST. Coated plates were incubated for 1 h at r.t. with sera that had been serially diluted in PBS containing 1% BSA, then washed with PBST, and incubated with HRP-conjugated IgG2C (PA1-29288, 1:15000) or HRP-conjugated IgG1 (PA1-74421, 1:6000) secondary antibodies at r.t. for 1 h. Plates were washed with 5× PBST and incubated with commercial TMB substrate for color development (Clinical Science Products Inc.). To stop the reaction, 2N H₂SO₄ was added and the plates were read at an optical density of 450 nm using an ELISA reader. End-point titers were calculated as the serum dilutions that emitted an optical density (O.D) greater than four times the background level (secondary antibody only), as described previously (17).

1.10 ELISpot Assay of Mouse Splenocytes

ELISpot assays to monitor cytokine levels in splenocytes stimulated with a SARS-CoV-2 spike peptide pool were performed essentially as described previously (82, 83). In brief, spleens were collected from immunized mice four weeks after vaccine boosting. We mixed 4×10⁵ splenocytes with a peptide pool of SARS-CoV-2 S protein sequences (Miltenyi Biotech, 130-126-700) at 1 μg/ml concentration in 100 μl medium (RPMI+10% FBS+1% PS), and then incubated them for 24 h at 37° C. in ELISpot plates (MABTECH) precoated with IFN-γ (3321-4AST-2), IL-2 (3441-4APW-2), IL-4 (3311-4APW-2), IL-6 (3361-4APW-2) or TNF-α (3511-4APW-2). Cells were then washed with 5x PBS and the ELIspots were developed according to the manufacturer's protocol and quantified using an AID vSpot machine.

1.11 Analyses of T Effector Memory (Tem) Cells

Flow cytometric analyses of Tem cells were performed as described previously (16) with minor modifications. Splenocytes were isolated from immunized mice at 4 weeks after vaccine boosting. After depleting red blood cells with Ammonium-Chloride-Potassium (ACK) lysis buffer, splenocytes were stimulated with 1 μg/ml of a SARS-CoV-2 spike-specific peptide pool (Miltenyi Biotech, 130-126-700) in medium (RPMI+10% FBS+1% PS) for 2 h at 37° C. The cells were subsequently washed twice with FACS buffer, and then incubated with an antibody cocktail including anti-CD3-PE/Cyanine7, anti-CD4-FITC, anti-CD8-Pacific blue, anti-CD44-PE and anti-CD62L-APC for 15 min on ice. The cells then underwent fluorescence-activated cell sorting (FACS), whereby CD4⁺ or CD8⁺ subpopulations were first gated from total splenocytes, and then further gated for CD44⁺CD62L⁻ as Tem cells. Dead cells were stained with eFluor 506 viability dye (eBioscience). Cells were acquired using a BD LSR II (BD Biosciences) flow cytometer and data analyses were performed with FlowJo 8.7 software.

1.12 Syrian Hamster Challenge Experiments

Syrian hamsters were immunized according to one of the three prime-boost vaccination regimens described above, anesthetized, with Zoletil-50 (50 mg/kg) and then intranasally (i.n) challenged with 1×10⁵ PFU of SARS-CoV-2 TCDC #4 (hCoV-19/Taiwan/4/2020, GISAID accession ID: EPI_ISL_411927) (lot: IBMS20200819, 8×10⁵ PFU/ml) in a volume of 125 μl. All animals were weighed daily after SARS-CoV-2 challenge. At 3 and 7 days post infection (d.p.i.), lungs were harvested for SARS-CoV-2 virus titer determination, viral RNA quantification and histopathological examination. Differences in body weight between experimental groups of animals were analyzed statistically using a two-tailed unpaired Student's t test.

1.13 Quantification of Viral Titers in Lung Tissues by Cell Culture Infection Assay

The middle, inferior, and post-caval lobes of hamsters at 3 and 7 days post challenge with SARS-CoV-2 were homogenized in 4 ml of DMEM with 2% FBS and 1% PS using a homogenizer. Tissue homogenate was centrifuged at 15,000 rpm for 5 min and the supernatant was collected for live virus titration. Briefly, 10-fold serial dilutions of each sample were added in quadruplicate onto a Vero E6 cell monolayer and incubated for 4 days. Cells were then fixed with 10% formaldehyde and stained with 0.5% crystal violet for 20 min. The plates were washed with tap water and scored for infection. The fifty-percent tissue culture infectious dose (TCID₅₀)/ml was calculated according to the Reed & Muench Method (81).

1.14 Real-Time RT-PCR for SARS-CoV-2 RNA Quantification

To measure the RNA levels of SARS-CoV-2, specific primers targeting nucleotides 26,141 to 26,253 of the SARS-CoV-2 envelope (E) gene were used for real-time RT-PCR, as described previously (84), forward primer E-Sarbeco-F1 (5′-ACAGGTACGTTAATAGTTAATAGCGT-3′) (SEQ ID NO: 6), reverse primer E-Sarbeco-R2 (5′-ATATTGCAGCAGTACGCACACA-3′) (SEQ ID NO: 7), probe E-Sarbeco-P1 (5′-FAM-ACACTAGCCATCCTTACTGCGCTTCG-BBQ-3′) (SEQ ID NO: 8). A total of 30 μl RNA solution was collected from each sample using an RNeasy Mini Kit (QIAGEN, Germany) according to the manufacturer's instructions. RNA sample (5 μl) was added into a total 25-μl mixture of the Superscript III one-step RT-PCR system with Platinum Taq Polymerase (Thermo Fisher Scientific, USA). The final reaction mix contained 400 nM of the forward and reverse primers, 200 nM probe, 1.6 mM deoxy-ribonucleoside triphosphate (dNTP), 4 mM magnesium sulfate, 50 nM ROX reference dye, and 1 μl of the enzyme mixture. Cycling conditions were performed using a one-step PCR protocol: 55° C. for 10 min for first-strand cDNA synthesis, followed by 3 min at 94° C. and 45 amplification cycles at 94° C. for 15 sec and 58° C. for 30 sec. Data was assessed using an Applied Biosystems 7500 Real-Time PCR System (Thermo Fisher Scientific). A synthetic 113-basepair oligonucleotide fragment was used as a qPCR standard to estimate copy numbers of the viral genome. The oligonucleotides were synthesized by Genomics BioSci & Tech Co. Ltd. (Taipei, Taiwan).

1.15 Histopathology

The left lung of each hamster at 3 and 7 days post challenge with SARS-CoV-2 was removed and fixed in 4% paraformaldehyde for 1 week. The lung samples were then embedded, sectioned, and stained with Hematoxylin and Eosin (H&E), followed by microscopic examination. Immunohistochemical staining was performed with a monoclonal rabbit anti-SARS-CoV/SARS-CoV-2 nucleocapsid (NP) antibody (1:1000, 40143-R001, Sino Biological), followed by incubation with Dako EnVision™+ System HRP. Brownish signals were subsequently developed upon addition of 3,3′ diaminobenzidine (DAB) and counterstained with hematoxylin. Images were photographed using a Zeiss Axioimager-Z1 microscope with 4× and 20× objective lenses.

1.16 Statistical Analyses

Statistical analyses were conducted using Student's t test in Prism (version 9) software (GraphPad). Statistical significance is represented as P values, with P values of less than 0.05 considered significant.

2. Results

We generated two different strains of recombinant vaccinia virus expressing SARS-CoV-2 spike protein. v-NY-S virus (BCRC970077) is replication-competent in mammalian cells, whereas the MVA-S strain is replication-restricted. We established three vaccination regimens in mice with different prime-boost combinations and show that all three generated high titers of neutralizing antibodies that blocked SARS-CoV-2 infections. These vaccination regimens also generated TH1-biased immune responses and effector memory CD8+ T cells. Finally, these vaccination regimens protected Syrian hamsters when subsequently challenged with SARS-CoV-2 virus.

2.1 Generation of Recombinant v-NY-S and MVA-S Viruses Expressing Full-Length SARS-CoV-2 S Protein.

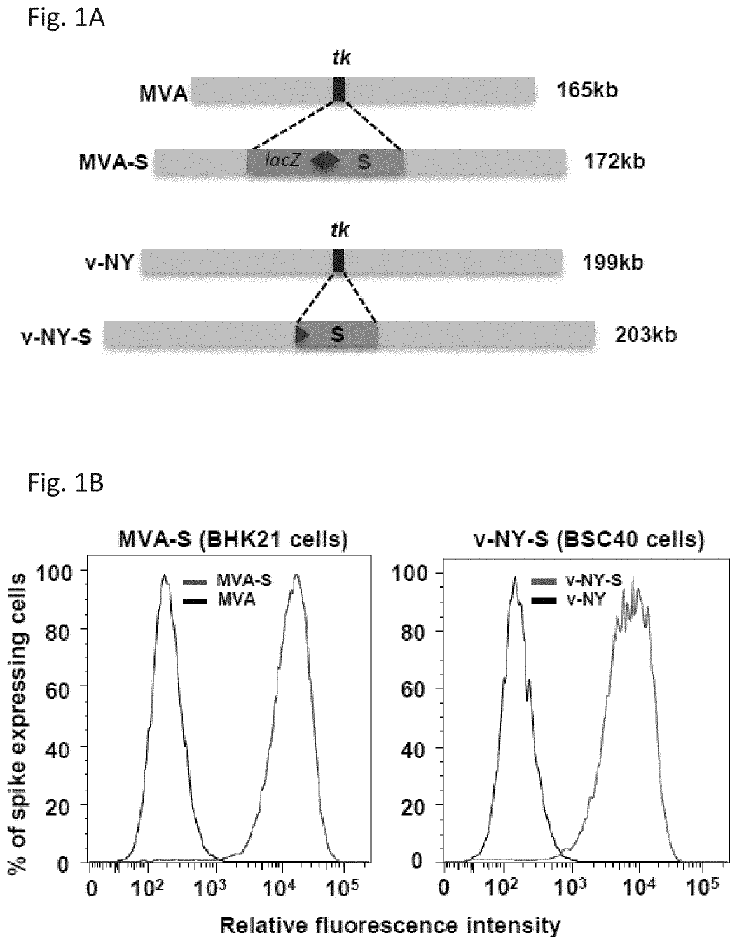

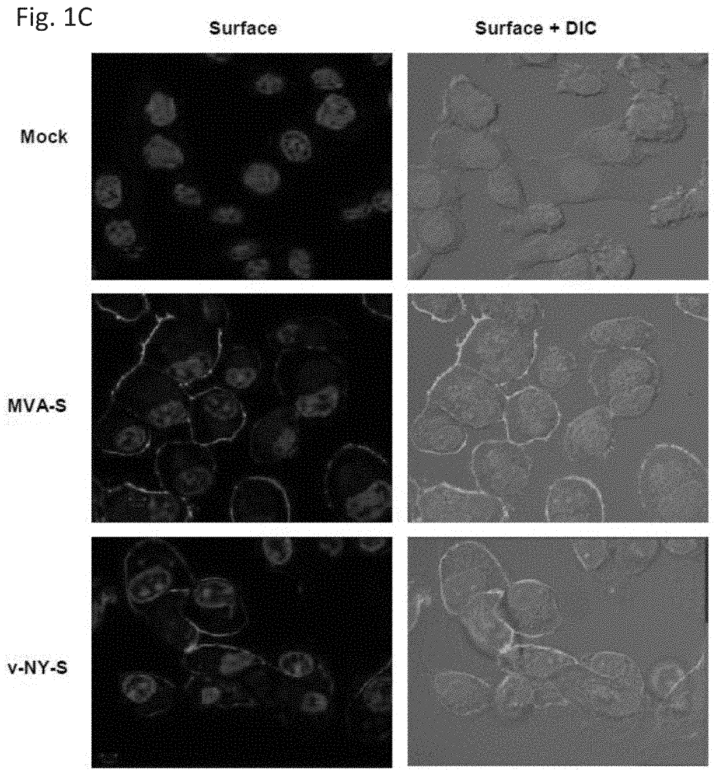

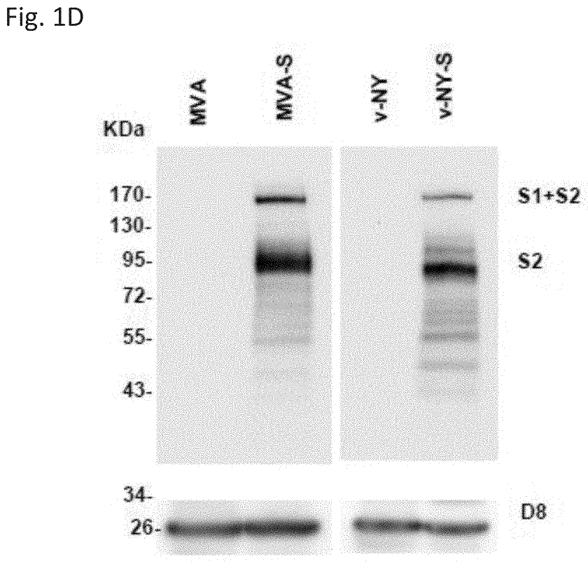

The recombinant vaccinia viruses MVA-S and v-NY-S were generated by inserting an ORF encoding full-length SARS-CoV-2 S protein (NC_045512) into the tk locus of the vaccinia virus strains MVA and v-NY, respectively (FIG. 1A). Cells infected with MVA-S and v-NY-S expressed high levels of SARS-CoV-2 S protein on cell surfaces, as revealed by flow cytometry (FIG. 1B) and immunofluorescence microscopy (FIG. 1C) analyses. Both full-length and processed forms of S protein were detected in immunoblots (FIG. 1D), confirming that the MVA-S and v-NY-S viruses stably expressed and processed SARS-CoV-2 S protein.

2.2 v-NY-S and MVA-S Prime-Boost Vaccination Regimens Generate Neutralizing Antibodies in Immunized C57BL/6 Mice.

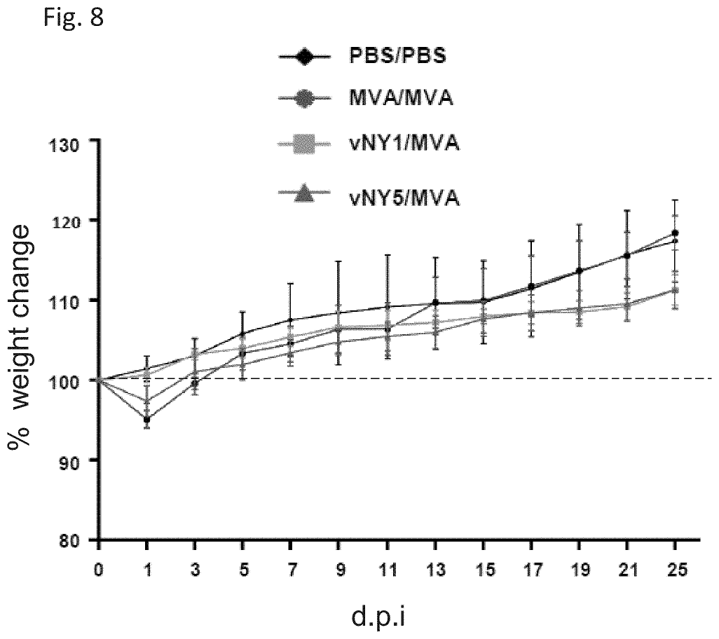

We designed three prime-boost vaccination regimens for the MVA-S and v-NY-S viruses (FIG. 2A). Control mice were primed and boosted with PBS buffer alone. For the first regimen (MVA/MVA), mice were primed i.m. with 5×10⁷ PFU/animal of MVA-S virus and boosted with 1×10⁷ PFU/animal of MVA-S. For the second regimen (v-NY1/MVA), mice were primed with v-NY-S by means of tail scarification (t.s.) at 1×10⁷ PFU/animal and then boosted i.m. with 1×10⁷ PFU/animal of MVA-S. For the third regimen (v-NY5/MVA), mice were primed by t.s. with v-NY-S at a higher titer of 5×10⁷ PFU/mouse and then boosted i.m. with 1×10⁷ PFU/animal of MVA-S. For each regimen, the mice were primed at day 0 and primary (1º) sera were collected 4 weeks later. These mice were then rested for 3 days, boosted, and then secondary (2º) sera were drawn 2 weeks later. In some experiments, spleens were harvested 4 weeks after boosting for T cell and cytokine analyses. The t.s. site of vaccinated mice healed well and the mice remained healthy without any loss of body weight (FIG. 8). 1º and 2º sera were collected from mice and flow cytometry revealed that they recognized SARS-CoV-2 S protein expressed from recombinant S-BAC baculovirus (FIG. 2B). Quantification (FIG. 2C) confirmed that anti-spike antibodies were specifically generated after primary immunization and that antibody titers were significantly enhanced after vaccine boosting. Mice primed with v-NY-S presented higher levels of anti-spike antibody compared to those primed with MVA-S (FIG. 2C). Immunoblot analyses (FIG. 1D) also revealed anti-spike antibody reactivity to recombinant S protein, consistent with our FACS data (FIG. 2C). We tested the neutralizing activity of 2º sera using a pseudotyped SARS-CoV-2 infection system (FIG. 2E, panel i) and a SARS-CoV-2 virus infection system (FIG. 2E, panel ii). Neutralization activity is presented as the reciprocal dilution of serum required for 50% inhibition of virus infection (ND₅₀). Our results show that all three regimens successfully generated high titers of neutralizing antibodies that inhibited SARS-CoV-2 S protein-mediated virus entry in both infection systems. Finally, we tested if our prime-boost vaccination regimens induced antibody responses by comparing mouse sera collected at 0.5 and 4.5 months after the MVA/MVA and vNY1/MVA regimens. Sera taken 4.5 months after boosting still contained 60-80% of spike-specific antibodies, as revealed by FACS analyses (FIG. 2F), and a pseudotyped SARS-CoV-2 virus infection assay demonstrated that they retained comparable neutralization activity to sera at 0.5 months (FIG. 2G), indicating these two vaccination regimens can elicit long-lived anti-spike antibody responses that have been shown to correlate with protection against SARS-CoV-2.

2.3 v-NY-S and MVA-S Immunization Generates a T_{H}1-Biased Immune Response in Mice.

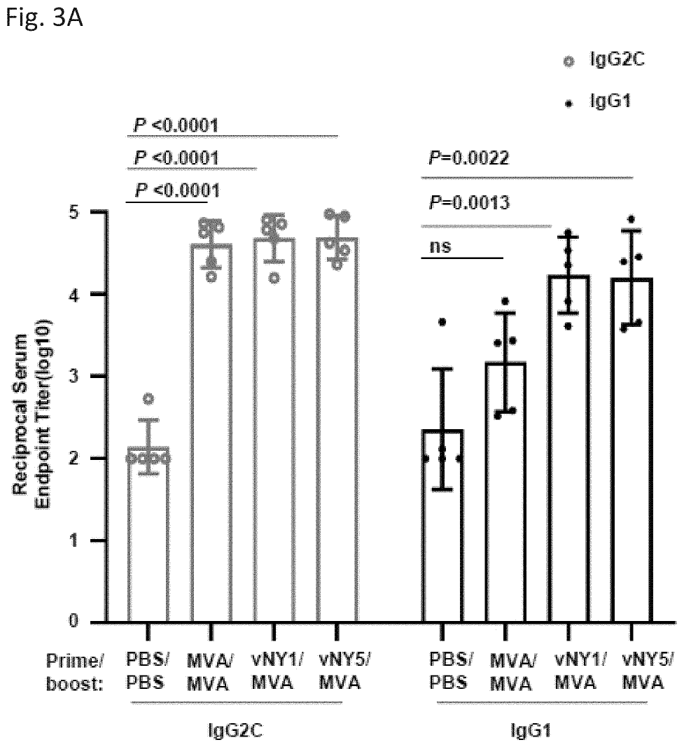

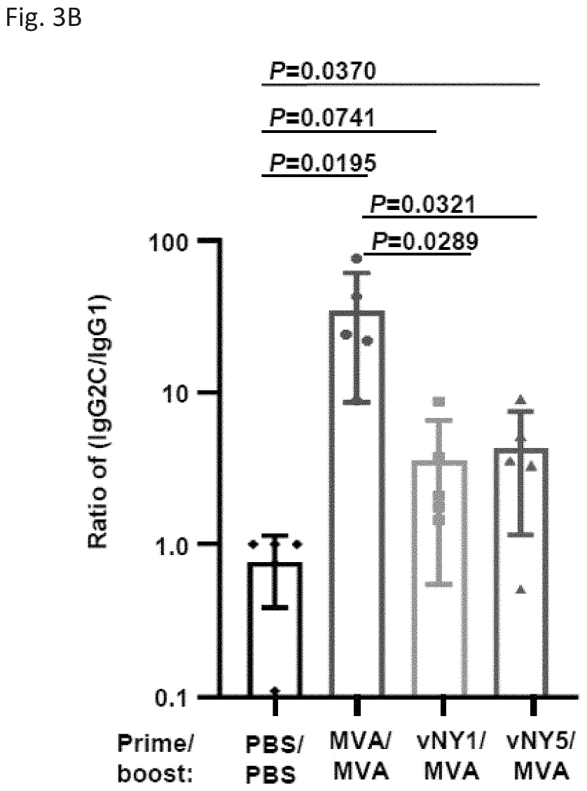

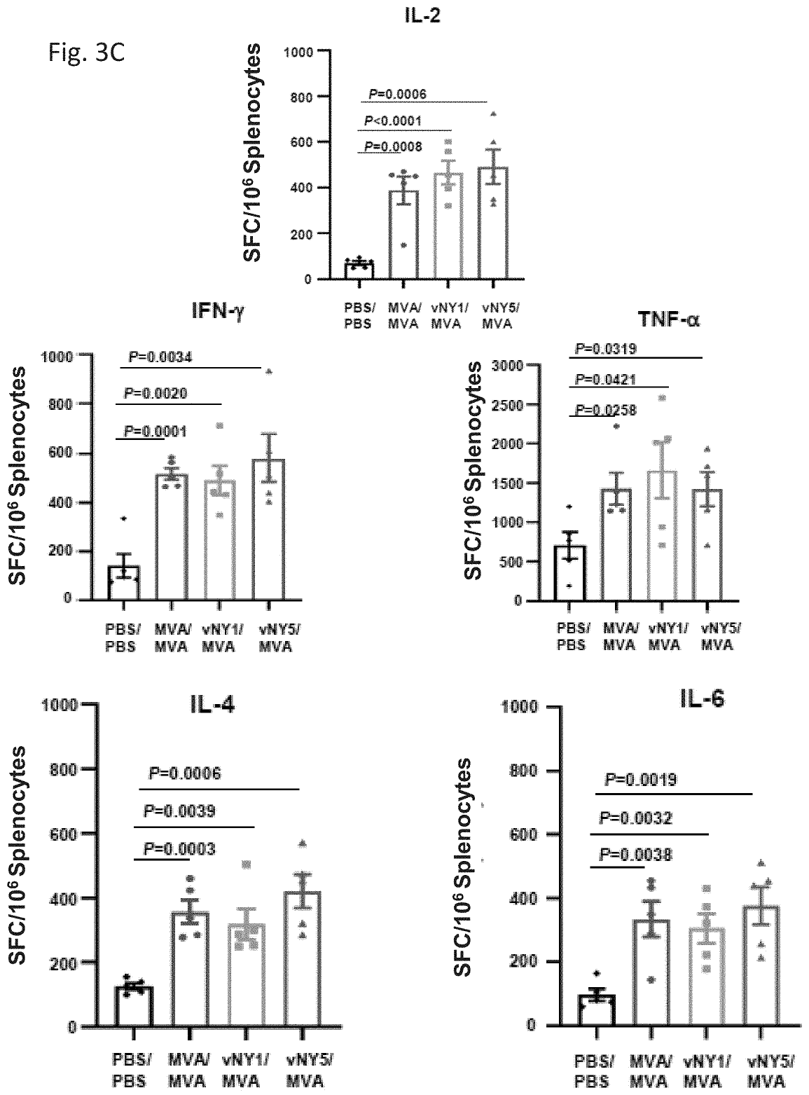

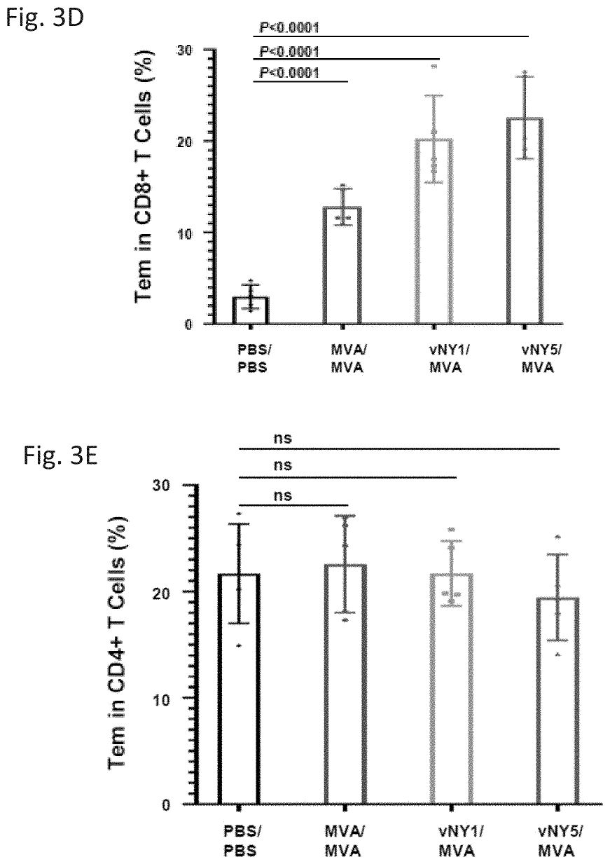

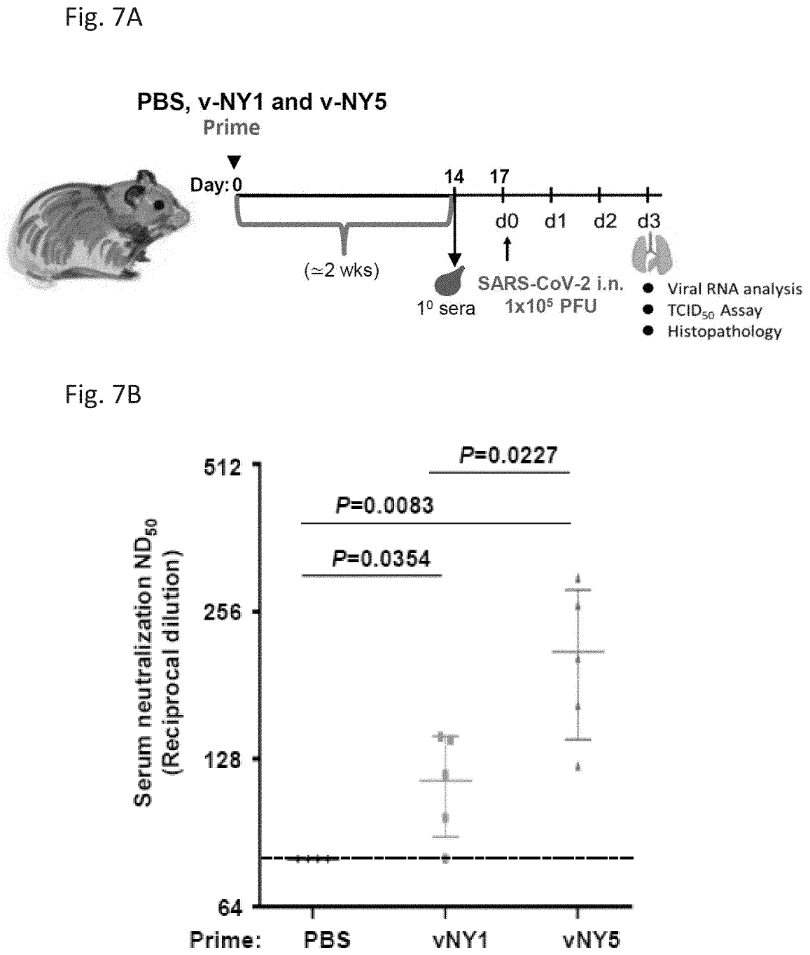

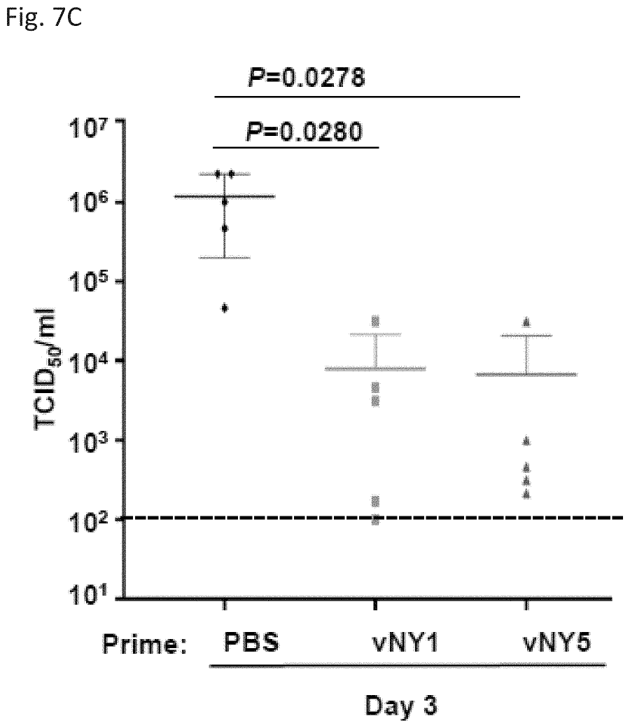

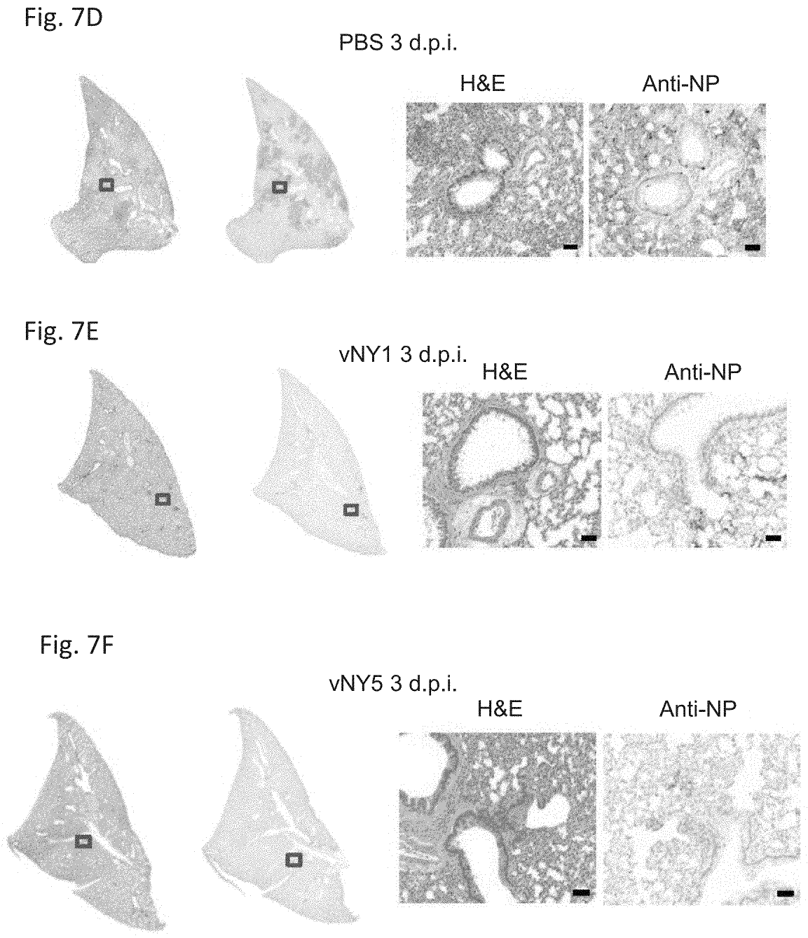

IFN-γ-producing T_{H}1 cells promote a B-cell class switch towards IgG2a/IgG2c, whereas IL-4-producing T_{H}2 cells promote a class switch towards IgG1 (49, 50). Therefore, a ratio of IgG2c (or IgG2a) to IgG1>1 is a good indicator of a T_{H}1-biased immune response, which is important for pathogen clearance. Accordingly, we used ELISA to measure levels of the IgG2c and IgG1 isotypes of anti-spike antibodies in C57BL/6 mouse sera collected after vaccination regimens (FIG. 3A). All three vaccination regimens induced production of the IgG2c and IgG1 isotypes (FIG. 3A) and with IgG2c/IgG1 ratios>1 (FIG. 3B), suggesting they had elicited a T_{H}1-biased immune response. We further in vitro-stimulated splenocytes from vaccinated mice with a SARS-CoV-2 spike peptide pool and then counted cells secreting T_{H}1 cytokines (IL-2, IFN-γ and TNF-α) and T_{H}2 cytokines (IL-4 and IL-6) (FIG. 3C) (51, 52). Consistently, we found that more cells secreted TNF-α and IFN-γ than IL-4 and IL-6, supporting that our three vaccination regimens triggered a T_{H}1-biased response. Furthermore, we investigated if our immunization regimens generated T effector memory (Tem) cells that are known to play a critical role in immune protection against secondary viral infections in lung tissue (53). Splenocytes isolated from mice 4 weeks after vaccination regimens were incubated with a SARS-CoV-2 spike peptide pool for 2 h and then analyzed by flow cytometry (FIGS. 3D & 3E), which revealed that all three regimens resulted in significantly increased numbers of FIG. 7F (FIG. 3D), but not CD4⁺ Tem cells (FIG. 3E), in spleen tissue.

2.4 v-NY-S and MVA-S Immunization Generated Neutralizing Antibodies in Immunized Syrian Hamsters.

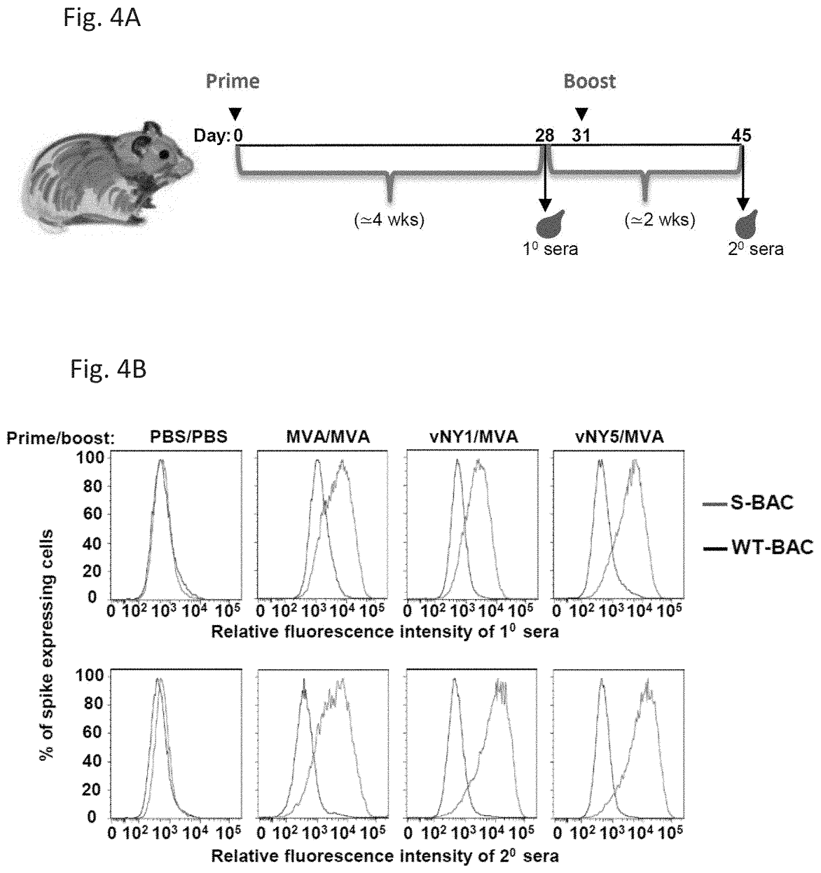

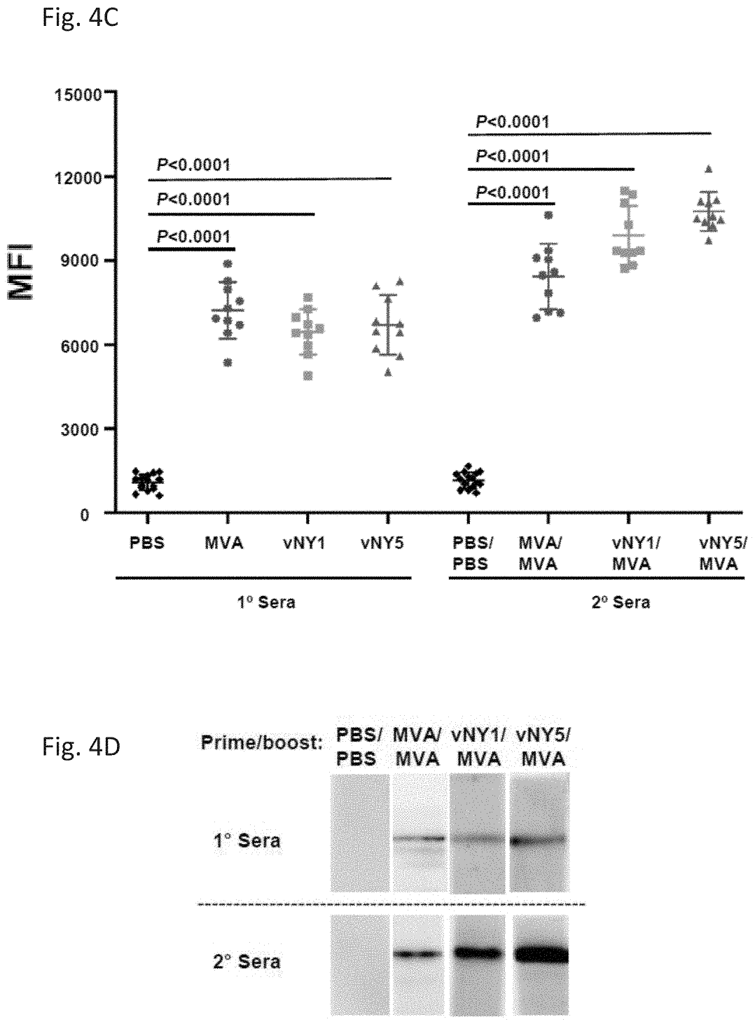

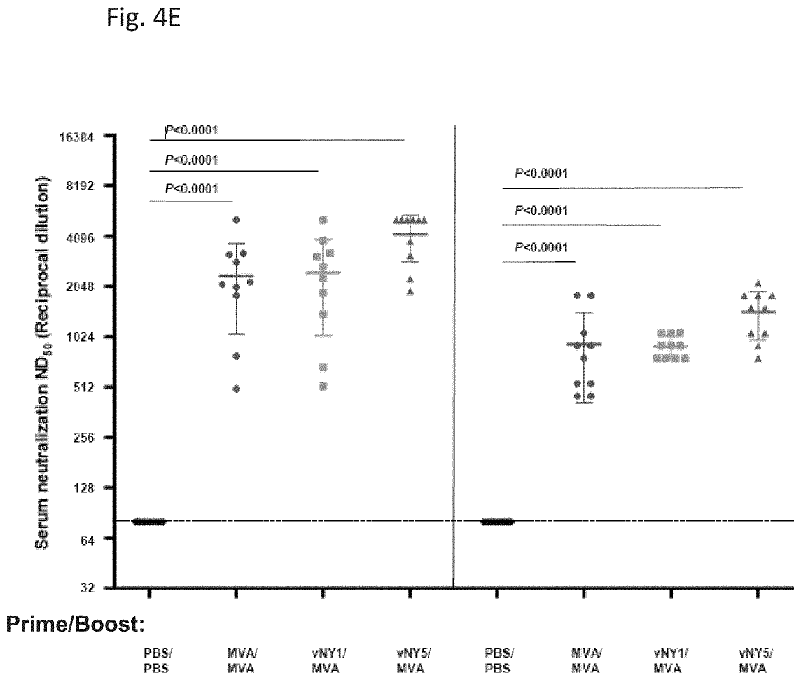

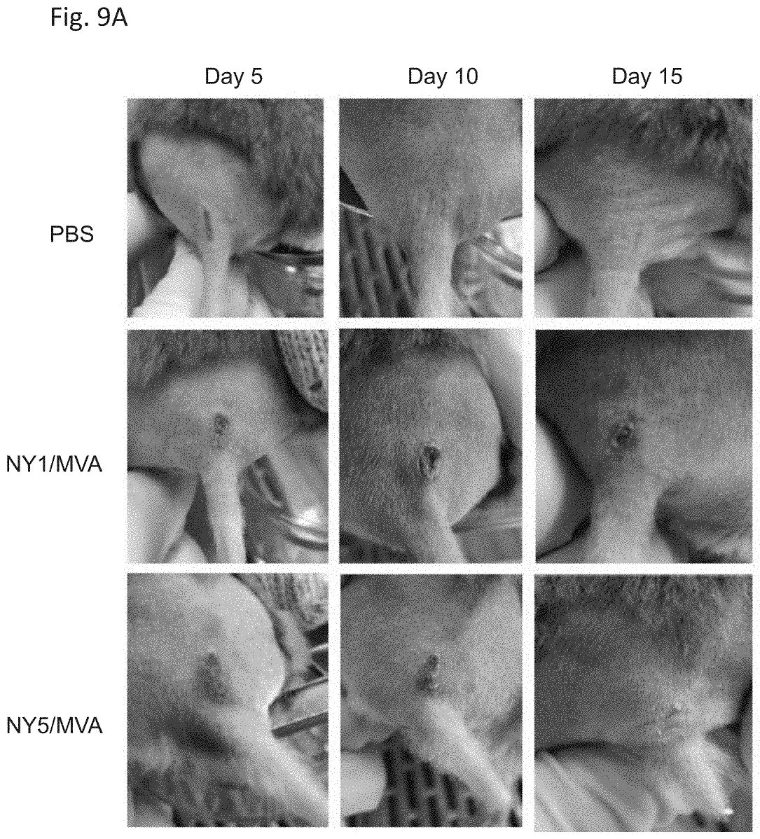

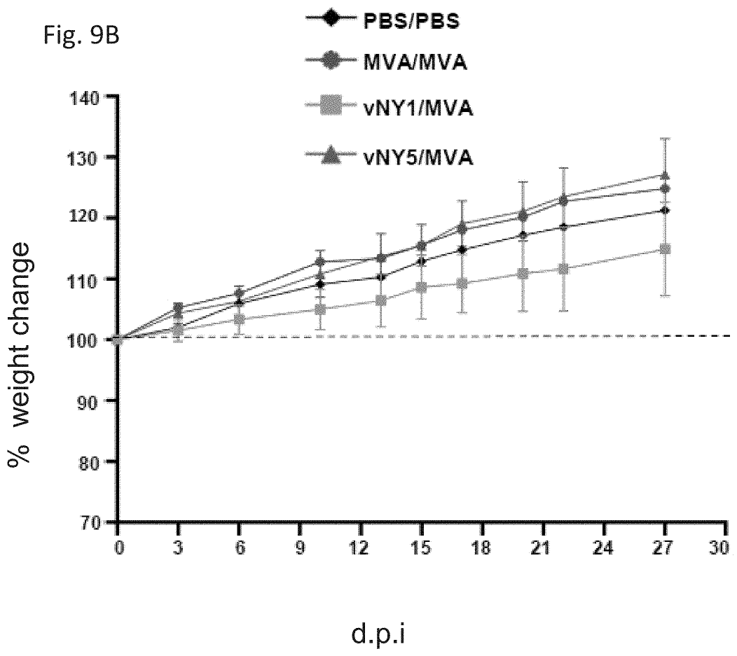

C57BL/6 mice are not susceptible to SARS-CoV-2 infection, whereas Syrian hamsters serve as an appropriate animal model of respiratory infection by SARS-CoV-2 in human (54-65). We subjected Syrian hamsters to the same prime-boost vaccination regimens, i.e., MVA/MVA, vNY1/MVA and vNYS/MVA (FIG. 4A) as applied to mice (FIG. 2A), except that we used a skin scarification inoculation approach for hamsters. A small scar formed at the immunization site, which healed within two weeks (FIG. 9A), and the immunized hamsters remained healthy any did not exhibit weight loss (FIG. 9B). Primary and secondary sera collected from these immunized hamsters specifically recognized SARS-CoV-2 S protein expressed on cell surfaces (FIG. 4B). Quantification of all hamster sera by FACS demonstrated that boosting enhanced anti-spike antibody titers (FIG. 4C), which was confirmed by immunoblotting (FIG. 4D). Importantly, all three vaccination regimens generated anti-spike antibodies with high neutralization activity in both pseudovirus (FIG. 4E, panel i) and live SARS-CoV-2 virus infection assays (FIG. 4E, panel ii). Taken together, these data show that, as observed for mice, our vaccination regimens generated high antibody titers in hamsters that neutralized SARS-CoV-2 infection.

2.5 v-NY-S and MVA-S Immunization Reduce Lung Pathology in SARS-CoV-2-Infected Syrian Hamsters.

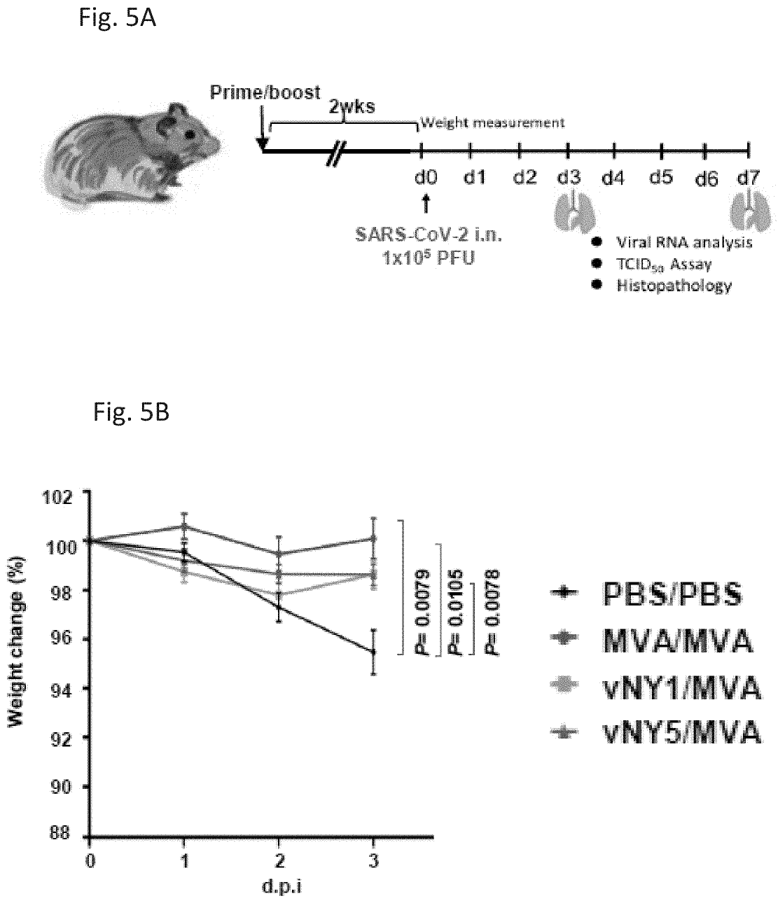

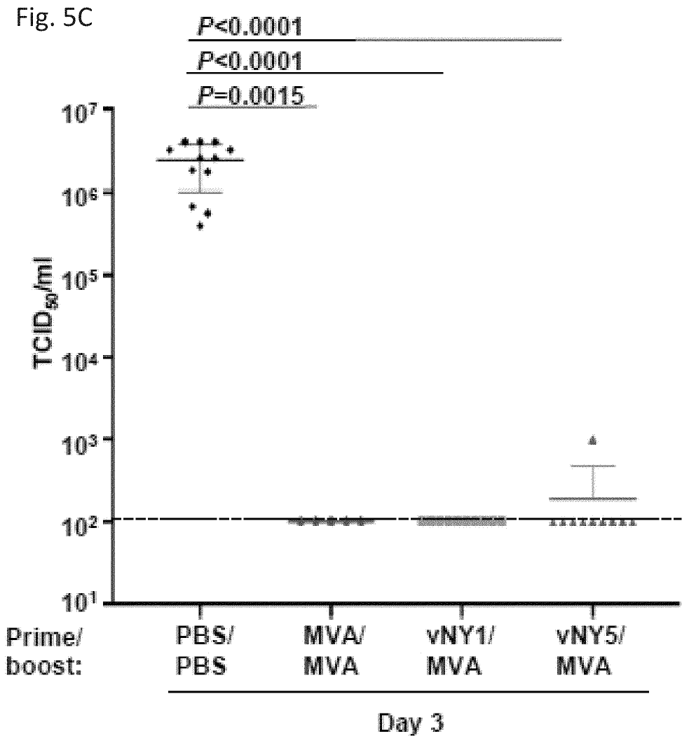

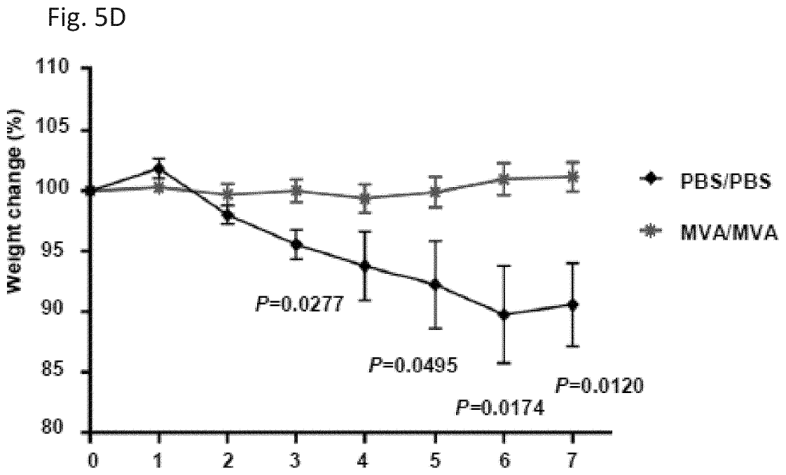

Next, we performed challenge experiments in hamsters 2 weeks after vaccine boosting by intranasally (i.n) inoculating 1×10⁵ PFU/animal of SARS-CoV-2 virus into each hamster and then measuring changes in body weight at 3 and 7 d.p.i. (FIG. 5A). Control hamsters (immunized with a PBS placebo) presented minor but detectable weight loss at 3 d.p.i., whereas those subjected to our vaccination regimens presented no obvious weight loss (FIG. 5B). A previous study has shown that SARS-CoV-2 infection of Syrian hamsters results in virus replication in lung tissue and that virus titers often peaked from 2-4 d.p.i. and gradually cleared by 7 d.p.i. (54, 66, 67). Therefore, we sacrificed hamsters at 3 d.p.i. and then measured SARS-CoV-2 virus titers in their lungs (FIG. 5C). None of the MVA/MVA- or vNY1/MVA-vaccinated hamsters presented detectable levels of SARS-CoV-2 virus in their lung tissue, whereas virus titers of up to ˜4×10⁶ TCID₅₀ were detected in the lungs of the placebo group (FIG. 5C). Moreover, no virus was detected in nine vNYS/MVA-immunized hamsters and only one such animal presented residual amounts of virus (<0.1% of the mean virus titer of the placebo group) (FIG. 5C).

We further explored the impact of our MVA/MVA regimen at 7 d.p.i. The weight loss of the placebo group was even more pronounced at 7 d.p.i. than at 3 d.p.i. (˜10-15%), whereas that of MVA/MVA-immunized hamsters remained unchanged (FIG. 5D). When we harvested lungs from immunized or placebo hamsters at 7 d.p.i. and measured SARS-CoV-2 virus titers and viral RNA levels, we found that no virus was detected in any of the hamsters (data not shown), but ˜10⁶ copies of viral RNA were detected in the lungs of the placebo group, whereas only ˜10² copies were detected in MVA/MVA-immunized hamsters (FIG. 5E).

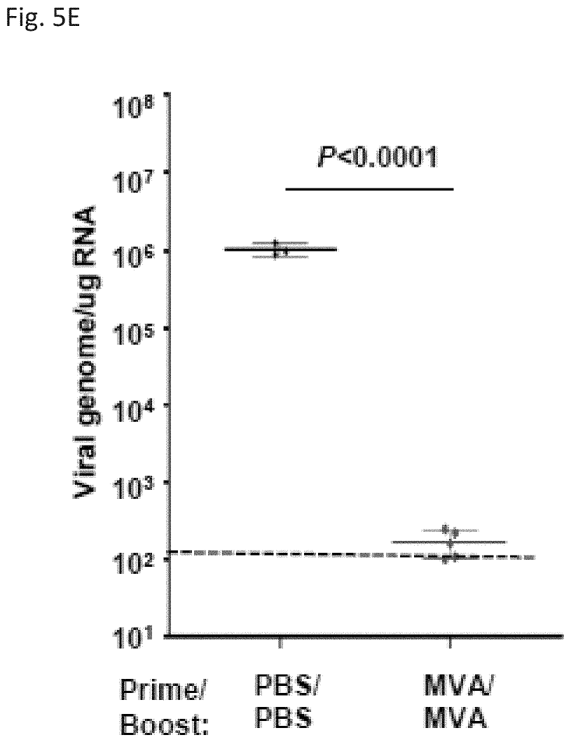

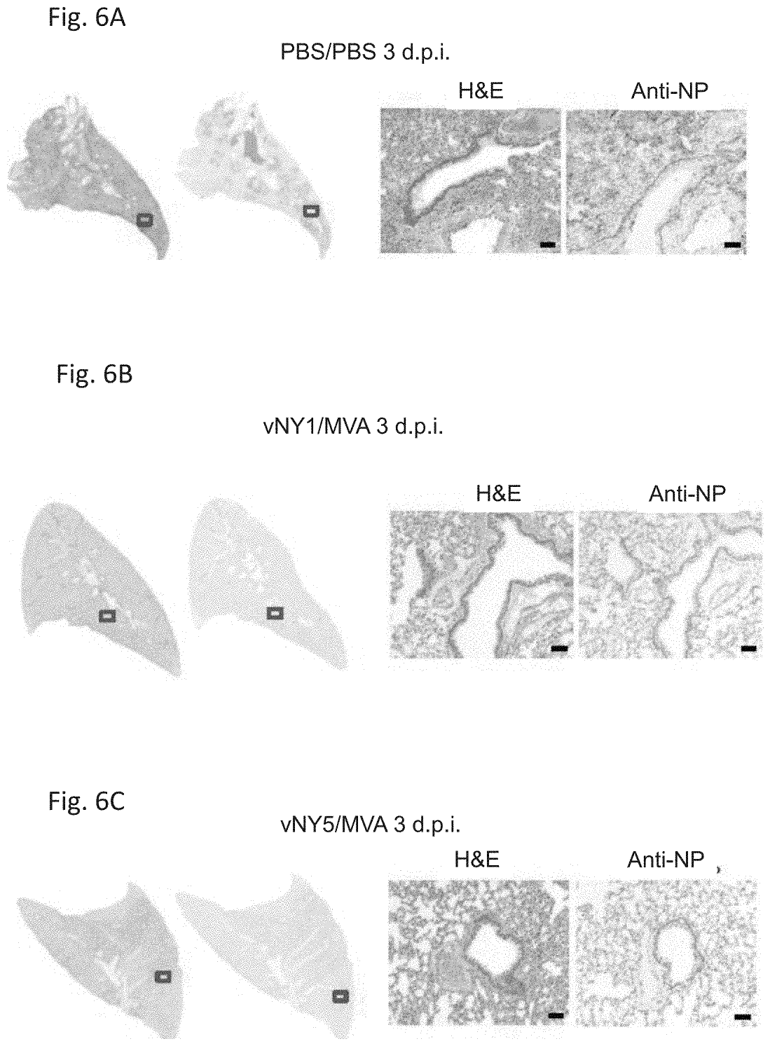

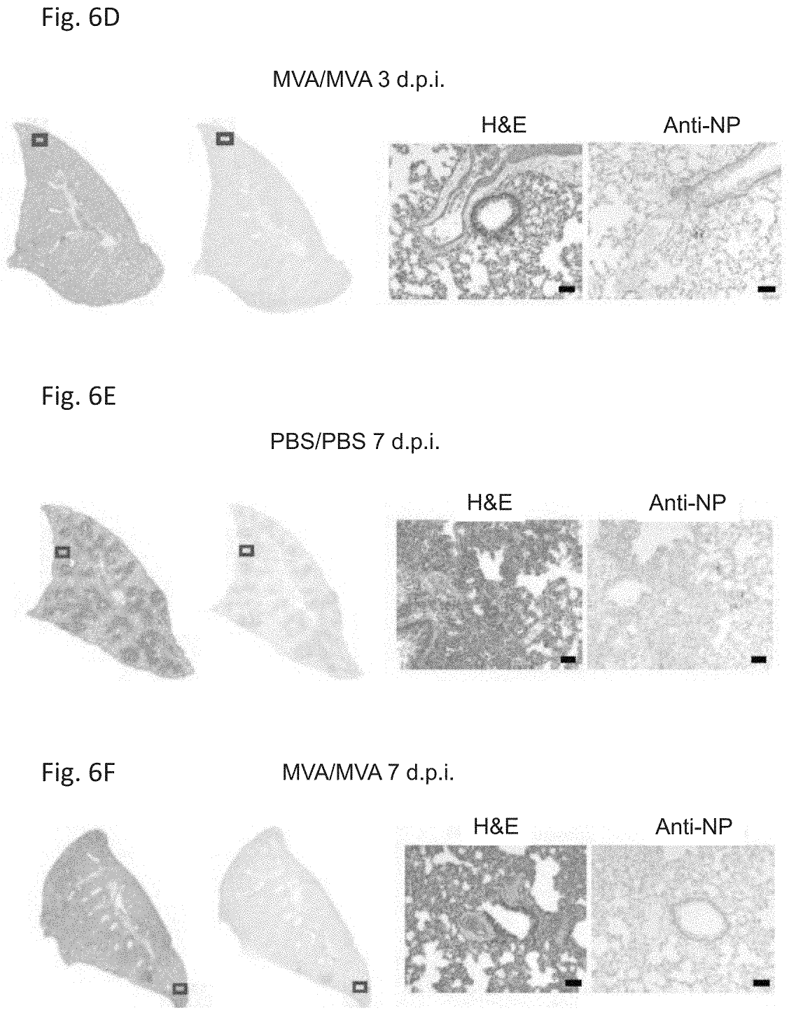

To further validate our findings, we removed the lungs of experimental hamsters at 3 and 7 d.p.i. and processed them for histological examination (FIG. 6). The lungs of placebo-infected hamsters at 3 d.p.i. presented diffuse congestion, shrinking of alveoli, hemorrhaging, and mononuclear cell infiltration. Moreover, bronchiolar epithelia vacuolization, necrosis and inflammatory exudates were also observed, and there was pronounced vasculitis and/or endothelialitis involving both medium and small blood vessels disrupted by a mixture of immune infiltrates. Immunostaining with an antibody against SARS-CoV-2 nucleocapsid (NP) protein revealed some areas of peribronchiolar immunoreactivity, mainly in the pneumocytes and less commonly in bronchiolar epithelial cells (FIG. 6A). Interestingly, despite the severe endothelial destruction observed by H&E staining, the SARS-CoV-2 NP antibody we deployed did not detect any positive viral-protein signal in the blood vessels of these placebo-infected hamsters. In contrast to the striking bronchointerstitial pneumonia observed in placebo-infected hamsters, there was only minimal to mild lung inflammation at 3 d.p.i. in the hamster groups subjected to the three vaccination regimens and SARS-CoV-2 NP protein signal was barely detectable (FIG. 6B, FIG. 6C, FIG. 6D).

We also examined the lung tissues of hamsters of the placebo and MVA/MVA groups at 7 d.p.i. (FIG. 6E & FIG. 6F). Profound type II pneumocyte hyperplasia was observed for the placebo-infection group, accompanied by mild to moderate neutrophilic infiltrate and numerous megakaryocytes centered on an obliterated bronchiole. Immunohistochemistry revealed weak but positive anti-NP antibody signal in pneumocytes at the periphery of bronchiole-centered lesions of placebo-infected hamsters (FIG. 6E). In contrast, the lungs of the MVA/MVA-infected group presented a less inflammatory phenotype at 7 d.p.i. and barely detectable anti-NP signal. Thus, taken together, our prime-boost vaccination regimens prevent SARS-CoV-2 viral spread in lung tissues and reduce inflammation and lung pathology.

2.6 Single Immunization with v-NY-S Partially Protects Syrian Hamsters from SARS-CoV-2 Infection.

We wished to establish if single-dose immunization with recombinant v-NY-S virus could provide protection against SARS-CoV-2 in Syrian hamsters. Hamsters were immunized with PBS (placebo), or 1×10⁷ or 5×10⁷ PFU/animal of v-NY-S by skin scarification and then sera were collected 2 weeks later (FIG. 7A). The sera were subjected to a SARS-CoV-2 pseudovirus neutralization assay, which showed that priming with v-NY-S alone generated neutralizing antibodies against SARS-CoV-2 virus in a dosage-dependent manner (FIG. 7B). Then we performed challenge experiments and monitored SARS-CoV-2 virus titers in lungs at 3 d.p.i. (FIG. 7C). Virus titers were ˜10⁶ PFU/animal in the placebo-infected group, but virus titers were >100-fold lower in hamsters subjected to single immunization with v-NY-S at either dosage (1×10⁷ or 5×10⁷ PFU/animal), showing that single immunization had already provided partial protection against SARS-CoV-2 infection. Upon removing lungs for histological examination, we observed that the placebo-infected group presented a severe pathological phenotype including diffuse congestion, shrinking of alveoli, hemorrhaging, and mononuclear cell infiltration (FIG. 7D). Moreover, immunostaining for SARS-CoV-2 NP protein also revealed widespread peribronchiolar immunoreactivity in the lungs of the placebo group (FIG. 7D). In contrast, the lung pathology of the vNY1-infected (FIG. 7E) and vNY5-infected (FIG. 7F) groups was much milder than observed for the placebo-infected group, displaying lower immune cell infiltration, rare epithelial degeneration and an absence of vasculitis/endothelialitis. Viral NP immune signal in lung tissues was also significantly lower in the vNY1 and vNY5 groups relative to the placebo group (rightmost panels in FIG. 7E & FIG. 7F).

3. Discussion

In this study, we demonstrated in preclinical models the safety and immunogenicity of recombinant vaccinia viruses expressing SARS-CoV-2 virus spike protein. All three of the prime-boost vaccination regimens with v-NY-S and MVA-S recombinants elicited strong and long-lasting neutralization antibody responses against SARS-CoV-2, and generated a T_{H}1-biased T cell response, which is beneficial for pathogen clearance. Importantly, we have demonstrated that our vaccination regimens protected Syrian hamsters (representing an appropriate animal model of SARS-CoV-2-induced respiratory disease in human) from weight loss, elicited rapid clearance of SARS-CoV-2 virus, and reduced immune infiltrates in lung tissue. Our study further explores the utility of the replication-competent v-NY strain, revealing that it could prove just as promising as the MVA strain in tackling SARS-CoV-2.

The MVA strain that is growth restricted in mammalian cells has been widely used in vaccine clinical studies due to its safety features. The v-NY strain has in vitro and in vivo characteristics similar to its parent virus, the New York City Board of Health strain of smallpox vaccine (44-47). Aspects of the v-NY strain have been characterized extensively, including plaque morphology, neutralization by vaccinia-specific monoclonal and polyclonal antisera, and its neurovirulence (44). It has been used to construct a recombinant virus expressing envelope glycoproteins of HIV-1 (HIVAC-1e) that has undergone FDA-approved early phase clinical trials (44). The availability of both recombinant MVA and v-NY viruses expressing SARS-CoV-2 S protein allows studies to determine whether genetic properties of the viral vector may modulate vaccine efficacy.

Most SARS-CoV-2 vaccines currently on the market require a two-dose prime-boost vaccination program to generate sufficient protective immunity (reviewed in(23) and references therein) except Ad26 vaccine which uses single immunization (55). Whether these vaccines would be sufficient for the control of the COVID pandemic is not clear. As the stability of currently available COVID vaccines depends on different degrees of the “cold chain” for storage and transportation, reliance on these vaccines alone for global vaccination may be difficult. In this context, the stability of the lyophilized smallpox vaccine, from which the v-NY vector was derived, may present certain advantages. Thus, our findings with the recombinant v-NY-S and MVA-S vaccine candidates in a prime-boost vaccination regimen may represent a very useful approach to tackling worldwide SARS-CoV-2 infections.

### Deposit of Microorganisms

The recombinant Vaccinia virus, v-NY-S, has been deposited on Jul. 14, 2021 with the Food Industry Research and Development Institute (FIRDI), Hsinchu city, Taiwan, and has been assigned the following accession number: BCRC970077; and also deposited on Jul. 13, 2022 with the Collection nationale de cultures de micro-organismes (CNCM), Paris, France, accession number: CNCM I-5857).

Sequence Information

SEQ ID NO: 1; Wuhan-Hu-1-YP_009724390.1
Spike a.a. sequence

| | |
|---|---|
| 1 | MFVFLVLLPL VSSQCVNLTT RTQLPPAYTN SFTRGVYYPD KVFRSSVLHS TQDLFLPFFS |
| 61 | NVTWFHAIHV SGTNGTKRFD NPVLPFNDGV YFASTEKSNI IRGWIFGTTL DSKTQSLLIV |
| 121 | NNATNVVIKV CEFQFCNDPF LGVYYHKNNK SWMESEFRVY SSANNCTFEY VSQPFLMDLE |
| 181 | GKQGNFKNLR EFVFKNIDGY FKIYSKHTPI NLVRDLPQGF SALEPLVDLP IGINITRFQT |
| 241 | LLALHRSYLT PGDSSSGWTA GAAAYYVGYL QPRTFLLKYN ENGTITDAVD CALDPLSETK |
| 301 | CTLKSFTVEK GIYQTSNFRV QPTESIVRFP NITNLCPFGE VFNATRFASV YAWNRKRISN |
| 361 | CVADYSVLYN SASFSTFKCY GVSPTKLNDL CFTNVYADSF VIRGDEVRQI APGQTGKIAD |
| 421 | YNYKLPDDFT GCVIAWNSNN LDSKVGGNYN YLYRLFRKSN LKPFERDIST EIYQAGSTPC |
| 481 | NGVEGFNCYF PLQSYGFQPT NGVGYQPYRV VVLSFELLHA PATVCGPKKS TNLVKNKCVN |
| 541 | FNFNGLTGTG VLTESNKKFL PFQQFGRDIA DTTDAVRDPQ TLEILDITPC SFGGVSVITP |
| 601 | GTNTSNQVAV LYQDVNCTEV PVAIHADQLT PTWRVYSTGS NVFQTRAGCL IGAEHVNNSY |
| 661 | ECDIPIGAGI CASYQTQTNS PRRARSVASQ SIIAYTMSLG AENSVAYSNN SIAIPTNFTI |
| 721 | SVTTEILPVS MTKTSVDCTM YICGDSTECS NLLLQYGSFC TQLNRALTGI AVEQDKNTQE |
| 781 | VFAQVKQIYK TPPIKDFGGF NFSQILPDPS KPSKRSFIED LLFNKVTLAD AGFIKQYGDC |
| 841 | LGDIAARDLI CAQKFNGLTV LPPLLTDEMI AQYTSALLAG TITSGWTFGA GAALQIPFAM |
| 901 | QMAYRFNGIG VTQNVLYENQ KLIANQFNSA IGKIQDSLSS TASALGKLQD VVNQNAQALN |
| 961 | TLVKQLSSNF GAISSVLNDI LSRLDKVEAE VQIDRLITGR LQSLQTYVTQ QLIRAAEIRA |
| 1021 | SANLAATKMS ECVLGQSKRV DFCGKGYHLM SFPQSAPHGV VFLHVTYVPA QEKNFTTAPA |
| 1081 | ICHDGKAHFP REGVFVSNGT HWFVTQRNFY EPQIITTDNT FVSGNCDVVI GIVNNTVYDP |
| 1141 | LQPELDSFKE ELDKYFKNHT SPDVDLGDIS GINASVVNIQ KEIDRLNEVA KNLNESLIDL |
| 1201 | QELGKYEQYI KWPWYIWLGF IAGLIAIVMV TIMLCCMTSC CSCLKGCCSC GSCCKFDEDD |
| 1261 | SEPVLKGVKL HYT |

SEQ ID NO: 2; α(B.1.1.7)-QWB50088.1
Spike a.a sequence

| | |
|---|---|
| 1 | MFVFLVLLPL VSSQCVNLTT RTQLPPAYTN SFTRGVYYPD KVFRSSVLHS TQDLFLPFFS |
| 6 | NVTWFHAISG TNGTKRFDNP VLPFNDGVYI ASTEKSNIIR GWIFGTTLDS KTQSLLIVNN |
| 121 | ATNVVIKVCE FQFCNDPFLG VYHKNNKSWM ESEFRVYSSA NNCTFEYVSQ PFLMDLEGKQ |
| 181 | GNFKNLREFV FKNIDGYFKI YSKHTPINLV RDLPQGFSAL EPLVDLPIGI NITRFQTLLA |
| 241 | LHRSYLTPGD SSSGWTAGAA AYYVGYLQPR TFLLKYNENG TITDAVDCAL DPLSETKCTL |
| 301 | KSFTVEKGIY QTSNFRVQPT ESIVRFPNIT NLCPFGEVFN ATRFASVYAW NRKRISNCVA |
| 361 | DYSVLYNSAS FSTFKCYGVS PTKLNDLCFT NVYADSFVIR GDEVRQIAPG QTGKIADYNY |
| 421 | KLPDDFTGCV IAWNSNNLDS KVGGNYNYLY RLFRKSNLKP FERDISTEIY QAGSTPCNGV |
| 481 | EGFNCYFPLQ SYGFQPTYGV GYQPYRVVVL SFELLHAPAT VCGPKKSTNL VKNKCVNFNE |
| 541 | NGLTGTGVLT ESNKKFLPFQ QFGRDIDDTT DAVRDPQTLE ILDITPCSFG GVSVITPGTN |
| 601 | TSNQVAVLYQ GVNCTEVPVA IHADQLTPTW RVYSTGSNVF QTRAGCLIGA EHVNNSYECD |
| 661 | IPIGAGICAS YQTQTNSHRR ARSVASQSII AYTMSLGAEN SVAYSNNSIA IPINFTISVT |
| 721 | TEILPVSMTK TSVDCTMYIC GDSTECSNLL LQYGSFCTQL NRALTGIAVE QDKNTQEVFA |
| 781 | QVKQIYKTPP IKDFGGFNFS QILPDPSKPS KRSFIEDLLF NKVTLADAGF IKQYGDCLGD |
| 841 | IAARDLICAQ KFNGLTVLPP LLTDEMIAQY TSALLAGTIT SGWTFGAGAA LQIPFAMQMA |
| 901 | YRFNGIGVTQ NVLYENQKLI ANQFNSAIGK IQDSLSSTAS ALGKLQDVVN QNAQALNTLV |
| 961 | KQLSSNFGAI SSVLNDILAR LDKVEAEVQI DRLITGRLQS LQTYVTQQLI RAAEIRASAN |
| 1021 | LAATKMSECV LGQSKRVDFC GKGYHLMSFP QSAPHGVVFL HVTYVPAQEK NFTTAPAICH |
| 1081 | DGKAHFPREG VFVSNGTHWF VTQRNFYEPQ IITTHNTFVS GNCDVVIGIV NNTVYDPLQP |
| 1141 | ELDSFKEELD KYFKNHTSPD VDLGDISGIN ASVVNIQKEI DRLNEVAKNL NESLIDLQEL |
| 1201 | GKYEQYIKWP WYIWLGFIAG LIAIVMVTIM LCCMTSCCSC LKGCCSCGSC CKFDEDDSEP |
| 1261 | VLKGVKLHYT |

SEQ ID NO: 3; β(B.1.351)-QWA53303.1
Spike a.a sequence.

| | |
|---|---|
| 1 | MFVFLVLLPL VSSQCVNLTT RTQLPPAYTN SFTRGVYYPD KVFRSSVLHS TQDLFLPFFS |
| 61 | NVTWFHAIHV SGTNGTKRFA NPVLPFNDGV YFASTEKSNI IRGWIFGTTL DSKTQSLLIV |
| 121 | NNATNVVIKV CEFQFCNDPF LGVYYHKNNK SWMESEFRVY SSANNCTFEY VSQPFLMDLE |
| 181 | GKQGNFKNLR EFVFKNIDGY FKIYSKHTPI NLVRGLPQGF SALEPLVDLP IGINITRFQT |
| 241 | LHRSYLTPGD SSSGWTAGAA AYYVGYLQPR TFLLKYNENG TITDAVDCAL DPLSETKCTL |
| 301 | KSFTVEKGIY QTSNFRVQPT ESIVRFPNIT NLCPFGEVFN ATRFASVYAW NRKRISNCVA |
| 361 | DYSVLYNSAS FSTFKCYGVS PTKLNDLCFT NVYADSFVIR GDEVRQIAPG QTGNIADYNY |
| 421 | KLPDDFTGCV IAWNSNNLDS KVGGNYNYLY RLFRKSNLKP FERDISTEIY QAGSTPCNGV |
| 481 | KGFNCYFPLQ SYGFQPTYGV GYQPYRVVVL SFELLHAPAT VCGPKKSTNL VKNKCVNFNF |
| 541 | NGLTGTGVLT ESNKKFLPFQ QFGRDIADTT DAVRDPQTLE ILDITPCSFG GVSVITPGTN |
| 601 | TSNQVAVLYQ GVNCTEVPVA IHADQLTPTW RVYSTGSNVF QTRAGCLIGA EHVNNSYECD |
| 661 | IPIGAGICAS YQTQTNSPRR ARSVASQSII AYTMSLGVEN SVAYSNNSIA IPTNFTISVT |
| 721 | TEILPVSMTK TSVDCTMYIC GDSTECSNLL LQYGSFCTQL NRALTGIAVE QDKNTQEVFA |
| 781 | QVKQIYKTPP IKDFGGFNFS QILPDPSKPS KRSFIEDLLF NKVTLADAGF IKQYGDCLGD |
| 841 | IAARDLICAQ KFNGLTVLPP LLTDEMIAQY TSALLAGTIT SGWTFGAGAA LQIPFAMQMA |
| 901 | YRFNGIGVTQ NVLYENQKLI ANQFNSAIGK IQDSLSSTAS ALGKLQDVVN QNAQALNTLV |
| 961 | KQLSSNFGAI SSVLNDILSR LDKVEAEVQI DRLITGRLQS LQTYVTQQLI RAAEIRASAN |
| 1021 | LAATKMSECV LGQSKRVDFC GKGYHLMSFP QSAPHGVVFL HVTYVPAQEK NFTTAPAICH |
| 1081 | DGKAHFPREG VFVSNGTHWF VTQRNFYEPQ IITTDNTFVS GNCDVVIGIV NNTVYDPLQP |
| 1141 | ELDSFKEELD KYFKNHTSPD VDLGDISGIN ASVVNIQKEI DRLNEVAKNL NESLIDLQEL |
| 1201 | GKYEQYIKWP WYIWLGFIAG LIAIVMVTIM LCCMTSCCSC LKGCCSCGSC CKFDEDDSEP |
| 1261 | VLKGVKLHYT |

SEQ ID NO: 4; γ(P.1)-QWB58007.1
Spike a.a sequence.

| | |
|---|---|
| 1 | MFVFLVLLPL VSSQCVNFTN RTQLPSAYTN SFTRGVYYPD KVFRSSVLHS TQDLFLPFFS |
| 61 | NVTWFHAIHV SGTNGTKRFD NPVLPFNDGV YFASTEKSNI IRGWIFGTTL DSKTQSLLIV |
| 121 | NNATNVVIKV CEFQFCNYPF LGVYYHKNNK SWMESEFRVY SSANNCTFEY VSQPFLMDLE |
| 181 | GKQGNFKNLS EFVFKNIDGY FKIYSKHTPI NLVRDLPQGF SALEPLVDLP IGINITRFQT |
| 241 | LLALHRSYLT PGDSSSGWTA GAAAYYVGYL QPRTFLLKYN ENGTITDAVD CALDPLSETK |
| 301 | CTLKSFTVEK GIYQTSNFRV QPTESIVRFP NITNLCPFGE VFNATRFASV YAWNRKRISN |
| 361 | CVADYSVLYN SASFSTFKCY GVSPTKLNDL CFTNVYADSF VIRGDEVRQI APGQTGTIAD |
| 421 | YNYKLPDDFT GCVIAWNSNN LDSKVGGNYN YLYRLFRKSN LKPFERDIST EIYQAGSTPC |
| 481 | NGVKGFNCYF PLQSYGFQPT YGVGYQPYRV VVLSFELLHA PATVCGPKKS TNLVKNKCVN |
| 541 | FNFNGLTGTG VLTESNKKFL PFQQFGRDIA DTTDAVRDPQ TLEILDITPC SFGGVSVITP |
| 601 | GTNTSNQVAV LYQGVNCTEV PVAIHADQLT PTWRVYSTGS NVFQTRAGCL IGAEYVNNSY |
| 661 | ECDIPIGAGI CASYQTQTNS PRRARSVASQ SIIAYTMSLG AENSVAYSNN SIAIPTNFTI |
| 721 | SVTTEILPVS MTKTSVDCTM YICGDSTECS NLLLQYGSFC TQLNRALTGI AVEQDKNTQE |
| 781 | VFAQVKQIYK TPPIKDFGGF NFSQILPDPS KPSKRSFIED LLFNKVTLAD AGFIKQYGDC |
| 841 | LGDIAARDLI CAQKFNGLTV LPPLLTDEMI AQYTSALLAG TITSGWTFGA GAALQIPFAM |
| 901 | QMAYRFNGIG VTQNVLYENQ KLIANQFNSA IGKIQDSLSS TASALGKLQD VVNQNAQALN |
| 961 | TLVKQLSSNF GAISSVLNDI LSRLDKVEAE VQIDRLITGR LQSLQTYVTQ QLIRAAEIRA |
| 1021 | SANLAAIKMS ECVLGQSKRV DFCGKGYHLM SFPQSAPHGV VFLHVTYVPA QEKNFTTAPA |
| 1081 | ICHDGKAHFP REGVFVSNGT HWFVTQRNFY EPQIITTDNT FVSGNCDVVI GIVNNTVYDP |
| 1141 | LQPELDSFKE ELDKYFKNHT SPDVDLGDIS GINASFVNIQ KEIDRLNEVA KNLNESLIDL |
| 1201 | QELGKYEQYI KWPWYIWLGF IAGLIAIVMV TIMLCCMTSC CSCLKGCCSC GSCCKFDEDD |
| 1261 | SEPVLKGVKL HYT |

SEQ ID NO: 5; δ(B.1.617.2)-QWB15066.1
Spike a.a sequence.

| | |
|---|---|
| 1 | MFVFLVLLPL VSSQCVNLRT RTQLPPAYTN SFTRGVYYPD KVFRSSVLHS TQDLFLPFFS |
| 61 | NVTWFHAIHV SGTNGTKRFD NPVLPFNDGV YFASTEKSNI IRGWIFGTTL DSKTQSLLIV |
| 121 | NNATNVVIKV CEFQFCNDPF LDVYYHKNNK SWMESGVYSS ANNCTFEYVS QPFLMDLEGK |
| 181 | QGNFKNLREF VFKNIDGYFK IYSKHTPINL VRDLPQGFSA LEPLVDLPIG INITRFQTLL |
| 241 | ALHRSYLTPG DSSSGWTAGA AAYYVGYLQP RTFLLKYNEN GTITDAVDCA LDPLSETKCT |
| 301 | LKSFTVEKGI YQTSNFRVQP TESIVRFPNI TNLCPFGEVF NATRFASVYA WNRKRISNCV |
| 361 | ADYSVLYNSA SFSTFKCYGV SPTKLNDLCF TNVYADSFVI RGDEVRQIAP GQTGKIADYN |
| 421 | YKLPDDFTGC VIAWNSNNLD SKVGGNYNYR YRLFRKSNLK PFERDISTEI YQAGSKPCNG |
| 481 | VEGFNCYFPL QSYGFQPTNG VGYQPYRVVV LSFELLHAPA TVCGPKKSTN LVKNKCVNFN |
| 541 | FNGLTGTGVL TESNKKFLPF QQFGRDIADT TDAVRDPQTL EILDITPCSF GGVSVITPGT |
| 601 | NTSNQVAVLY QGVNCTEVPV AIHADQLTPT WRVYSTGSNV FQTRAGCLIG AEHVNNSYEC |
| 661 | DIPIGAGICA SYQTQTNSRR RARSVASQSI IAYTMSLGAE NSVAYSNNSI AIPTNFTISV |
| 721 | TTEILPVSMT KTSVDCTMYI CGDSTECSNL LLQYGSFCTQ LNRALTGIAV EQDKNTQEVF |
| 781 | AQVKQIYKTP PIKDFGGFNF SQILPDPSKP SKRSFIEDLL FNKVTLADAG FIKQYGDCLG |
| 841 | DIAARDLICA QKFNGLTVLP PLLTDEMIAQ YTSALLAGTI TSGWTFGAGA ALQIPFAMQM |
| 901 | AYRFNGIGVT QNVLYENQKL IANQFNSAIG KIQDSLSSTA SALGKLQNVV NQNAQALNTL |
| 961 | VKQLSSNFGA ISSVLNDILS RLDKVEAEVQ IDRLITGRLQ SLQTYVTQQL IRAAEIRASA |
| 1021 | NLAATKMSEC VLGQSKRVDF CGKGYHLMSF PQSAPHGVVF LHVTYVPAQE KNFTTAPAIC |
| 1081 | HDGKAHFPRE GVFVSNGTHW FVTQRNFYEP QIITTDNTFV SGNCDVVIGI VNNTVYDPLQ |
| 1141 | PELDSFKEEL DKYFKNHTSP DVDLGDISGI NASVVNIQKE IDRLNEVAKN LNESLIDLQE |
| 1201 | LGKYEQYIKW PWYIWLGFIA GLIAIVMVTI MLCCMTSCCS CLKGCCSCGS CCKFDEDDSE |
| 1261 | PVLKGVKLHY T |

| Variant | Mutation(S) in Spike | Note* |
|---|---|---|
| WT | NA | — |
| α (B.1.1.7) | 69del, 70del, 144del, N501Y, A570D, | VOC |
| | D614G, P681H, T716I, S982A, D1118H | |
| β (B.1.351) | D80A, D215G, 241del, 242del, 243del, | VOC |
| | K417N, E484K, N501Y, D614G, A701V | |
| γ (P.1) | L18F, T20N, P26S, D138Y, R190S, K417T, | VOC |
| | E484K, N501Y, D614G, H655Y, T1027I | |
| δ (B.l.617.2) | T19R, (G142D), 156del, 157del, R158G, | VOI |
| | L452R, T478K, D614G, P681R, D950N | |

VOC: Variant of concern; VOI: Variant of Interest
*Based on the C DC website (https://www.cdc.gov/coronavirus/2019-ncov/variants/va riant-info.html)

| SARS-CoV-2 | Spike protein |
|---|---|
| Virus strains | (NCBI ID) |
| Wuhan-Hu-1 | YP_009724390.1 |
| α (B.1.1.7) | QWB50088.1 |
| β (B.1.351) | QWA53303.1 |
| γ (P.1) | QWB58007.1 |
| δ (B.l.617.2) | QWB15066.1 |

### REFERENCES

1. Zhou P, Yang X L, Wang X G, Hu B, Zhang L, Zhang W, et al. A pneumonia outbreak associated with a new coronavirus of probable bat origin. Nature. 2020; 579(7798):270-3.
2. Coronaviridae Study Group of the International Committee on Taxonomy of V. The species Severe acute respiratory syndrome-related coronavirus: classifying 2019-nCoV and naming it SARS-CoV-2. Nat Microbiol. 2020; 5(4):536-44.
3. Drosten C, Gunther S, Preiser W, van der Werf S, Brodt H R, Becker S, et al. Identification of a novel coronavirus in patients with severe acute respiratory syndrome. N Engl J Med. 2003; 348(20):1967-76.
4. Zaki A M, van Boheemen S, Bestebroer T M, Osterhaus A D, Fouchier R A. Isolation of a novel coronavirus from a man with pneumonia in Saudi Arabia. N Engl J Med. 2012; 367(19):1814-20.
5. Petersen E, Koopmans M, Go U, Hamer D H, Petrosillo N, Castelli F, et al. Comparing SARS-CoV-2 with SARS-CoV and influenza pandemics. Lancet Infect Dis. 2020; 20(9): e23 8-e44.
6. Chan J F, Kok K H, Zhu Z, Chu H, To K K, Yuan S, et al. Genomic characterization of the 2019 novel human-pathogenic coronavirus isolated from a patient with atypical pneumonia after visiting Wuhan. Emerg Microbes Infect. 2020; 9(1):221-36.
7. Hu B, Guo H, Zhou P, Shi Z L. Characteristics of SARS-CoV-2 and COVID-19. Nat Rev Microbiol. 2020.
8. Shang J, Wan Y, Luo C, Ye G, Geng Q, Auerbach A, et al. Cell entry mechanisms of SARS-CoV-2. Proc Natl Acad Sci USA. 2020; 117(21):11727-34.
9. Yang J, Petitjean S J L, Koehler M, Zhang Q, Dumitru A C, Chen W, et al. Molecular interaction and inhibition of SARS-CoV-2 binding to the ACE2 receptor. Nat Commun. 2020; 11(1):4541.
10. Ke Z, Oton J, Qu K, Cortese M, Zila V, McKeane L, et al. Structures and distributions of SARS-CoV-2 spike proteins on intact virions. Nature. 2020; 588(7838):498-502.
11. Hoffmann M, Kleine-Weber H, Schroeder S, Kruger N, Herrler T, Erichsen S, et al. SARS-CoV-2 Cell Entry Depends on ACE2 and TMPRSS2 and Is Blocked by a Clinically Proven Protease Inhibitor. Cell. 2020; 181(2):271-80 e8.
12. Dai L, Gao G F. Viral targets for vaccines against COVID-19. Nat Rev Immunol. 2020.
13. Ju B, Zhang Q, Ge J, Wang R, Sun J, Ge X, et al. Human neutralizing antibodies elicited by SARS-CoV-2 infection. Nature. 2020; 584(7819):115-9.
14. Grifoni A, Sidney J, Zhang Y, Scheuermann R H, Peters B, Sette A. Candidate Targets for Immune Responses to 2019-Novel Coronavirus (nCoV): Sequence Homology- and Bioinformatic-Based Predictions. SSRN. 2020:3541361.
15. Grifoni A, Sidney J, Zhang Y, Scheuermann R H, Peters B, Sette A. A Sequence Homology and Bioinformatic Approach Can Predict Candidate Targets for Immune Responses to SARS-CoV-2. Cell Host Microbe. 2020; 27(4):671-80 e2.
16. Zhang N N, Li X F, Deng Y Q, Zhao H, Huang Y J, Yang G, et al. A Thermostable mRNA Vaccine against COVID-19. Cell. 2020; 182(5):1271-83 e16.
17. Corbett K S, Edwards D K, Leist S R, Abiona O M, Boyoglu-Barnum S, Gillespie R A, et al. SARS-CoV-2 mRNA vaccine design enabled by prototype pathogen preparedness. Nature. 2020; 586(7830):567-71.
18. Vogel A B, Kanevsky I, Che Y, Swanson K A, Muik A, Vormehr M, et al. BNT162b vaccines protect rhesus macaques from SARS-CoV-2. Nature. 2021.
19. Mercado N B, Zahn R, Wegmann F, Loos C, Chandrashekar A, Yu J, et al. Single-shot Ad26 vaccine protects against SARS-CoV-2 in rhesus macaques. Nature. 2020; 586(7830):583-8.
20. Logunov D Y, Dolzhikova I V, Zubkova O V, Tukhvatulin A I, Shcheblyakov D V, Dzharullaeva A S, et al. Safety and immunogenicity of an rAd26 and rAdS vector-based heterologous prime-boost COVID-19 vaccine in two formulations: two open, non-randomised phase 1/2 studies from Russia. Lancet. 2020; 396(10255):887-97.
21. van Doremalen N, Lambe T, Spencer A, Belij-Rammerstorfer S, Purushotham J N, Port J R, et al. ChAdOx1 nCoV-19 vaccine prevents SARS-CoV-2 pneumonia in rhesus macaques. Nature. 2020; 586(7830):578-82.
22. Krammer F. SARS-CoV-2 vaccines in development. Nature. 2020; 586(7830):516-27.
23. Klasse P J, Nixon D F, Moore J P. Immunogenicity of clinically relevant SARS-CoV-2 vaccines in nonhuman primates and humans. Sci Adv. 2021; 7(12).
24. Greinacher A, Thiele T, Warkentin T E, Weisser K, Kyrle P A, Eichinger S. Thrombotic Thrombocytopenia after ChAdOx1 nCov-19 Vaccination. N Engl J Med. 2021.
25. Parrino J, Graham B S. Smallpox vaccines: Past, present, and future. J Allergy Clin Immunol. 2006; 118(6):1320-6.
26. Mayr A. Smallpox vaccination and bioterrorism with pox viruses. Comp Immunol Microbiol Infect Dis. 2003; 26(5-6):423-30.
27. Kreijtz J H, Goeijenbier M, Moesker F M, van den Dries L, Goeijenbier S, De Gruyter H L, et al. Safety and immunogenicity of a modified-vaccinia-virus-Ankara-based influenza A H5N1 vaccine: a randomised, double-blind phase 1/2a clinical trial. Lancet Infect Dis. 2014; 14(12):1196-207.
28. Verheust C, Goossens M, Pauwels K, Breyer D. Biosafety aspects of modified vaccinia virus Ankara (MVA)-based vectors used for gene therapy or vaccination. Vaccine. 2012; 30(16):2623-32.
29. Thompson M, Heath SL, Sweeton B, Williams K, Cunningham P, Keele B F, et al. DNA/MVA Vaccination of HIV-1 Infected Participants with Viral Suppression on Antiretroviral Therapy, followed by Treatment Interruption: Elicitation of Immune Responses without Control of Re-Emergent Virus. Plos One. 2016; 11(10):e0163164.
30. Koch T, Dahlke C, Fathi A, Kupke A, Krahling V, Okba N M A, et al. Safety and immunogenicity of a modified vaccinia virus Ankara vector vaccine candidate for Middle East respiratory syndrome: an open-label, phase 1 trial. Lancet Infect Dis. 2020; 20(7): 827-38.
31. Jacobs B L, Langland J O, Kibler K V, Denzler K L, White S D, Holechek S A, et al. Vaccinia virus vaccines: past, present and future. Antiviral Res. 2009; 84(1):1-13.
32. Walsh S R, Dolin R. Vaccinia viruses: vaccines against smallpox and vectors against infectious diseases and tumors. Expert Rev Vaccines. 2011; 10(8):1221-40.
33. Verardi P H, Titong A, Hagen C J. A vaccinia virus renaissance: new vaccine and immunotherapeutic uses after smallpox eradication. Hum Vaccin Immunother. 2012; 8(7):961-70.
34. Heo J, Reid T, Ruo L, Breitbach C J, Rose S, Bloomston M, et al. Randomized dose-finding clinical trial of oncolytic immunotherapeutic vaccinia JX-594 in liver cancer. Nat Med. 2013; 19(3):329-36.
35. Zeh H J, Downs-Canner S, McCart J A, Guo Z S, Rao U N, Ramalingam L, et al. First-in-man study of western reserve strain oncolytic vaccinia virus: safety, systemic spread, and antitumor activity. Mol Ther. 2015; 23(1):202-14.
36. Downs-Canner S, Guo Z S, Ravindranathan R, Breitbach C J, O'Malley M E, Jones H L, et al. Phase 1 Study of Intravenous Oncolytic Poxvirus (vvDD) in Patients With Advanced Solid Cancers. Mol Ther. 2016; 24(8):1492-501.
37. Lauer U M, Schell M, Beil J, Berchtold S, Koppenhofer U, Glatzle J, et al. Phase I Study of Oncolytic Vaccinia Virus GL-ONC1 in Patients with Peritoneal Carcinomatosis. Clin Cancer Res. 2018; 24(18):4388-98.
38. Guo Z S, Lu B, Guo Z, Giehl E, Feist M, Dai E, et al. Vaccinia virus-mediated cancer immunotherapy: cancer vaccines and oncolytics. J Immunother Cancer. 2019; 7(1):6.
39. Minev B R, Lander E, Feller J F, Berman M, Greenwood B M, Minev I, et al. First-in-human study of TK-positive oncolytic vaccinia virus delivered by adipose stromal vascular fraction cells. J Transl Med. 2019; 17(1):271.
40. Kennedy J S, Gurwith M, Dekker C L, Frey S E, Edwards K M, Kenner J, et al. Safety and immunogenicity of LC16m8, an attenuated smallpox vaccine in vaccinia-naive adults. J Infect Dis. 2011; 204(9):1395-402.
41. Garcia-Arriaza J, Garaigorta U, Perez P, Lazaro-Frias A, Zamora C, Gastaminza P, et al. COVID-19 vaccine candidates based on modified vaccinia virus Ankara expressing the SARS-CoV-2 spike induce robust T- and B-cell immune responses and full efficacy in mice. J Virol. 2021.
42. Liu R, Americo J L, Cotter C A, Earl P L, Erez N, Peng C, et al. One or two injections of MVA-vectored vaccine shields hACE2 transgenic mice from SARS-CoV-2 upper and lower respiratory tract infection. Proc Natl Acad Sci USA. 2021; 118(12).
43. Routhu N K, Cheedarla N, Gangadhara S, Bollimpelli V S, Boddapati A K, Shiferaw A, et al. A modified vaccinia Ankara vector-based vaccine protects macaques from SARS-CoV-2 infection, immune pathology, and dysfunction in the lungs. Immunity. 2021; 54(3):542-56 e9.
44. Cooney E L, Collier A C, Greenberg P D, Coombs R W, Zarling J, Arditti D E, et al. Safety of and immunological response to a recombinant vaccinia virus vaccine expressing HIV envelope glycoprotein. Lancet. 1991; 337(8741):567-72.
45. Graham B S, Belshe R B, Clements M L, Dolin R, Corey L, Wright P F, et al. Vaccination of vaccinia-naive adults with human immunodeficiency virus type 1 gp160 recombinant vaccinia virus in a blinded, controlled, randomized clinical trial. The AIDS Vaccine Clinical Trials Network. J Infect Dis. 1992; 166(2):244-52.
46. Cooney E L, McElrath M J, Corey L, Hu S L, Collier A C, Arditti D, et al. Enhanced immunity to human immunodeficiency virus (HIV) envelope elicited by a combined vaccine regimen consisting of priming with a vaccinia recombinant expressing HIV envelope and boosting with gp160 protein. Proc Natl Acad Sci USA. 1993; 90(5):1882-6.
47. Graham B S, Matthews T J, Belshe R B, Clements M L, Dolin R, Wright P F, et al. Augmentation of human immunodeficiency virus type 1 neutralizing antibody by priming with gp160 recombinant vaccinia and boosting with rgp160 in vaccinia-naive adults. The NIAID AIDS Vaccine Clinical Trials Network. J Infect Dis. 1993; 167(3):533-7.
48. Sutter G, Moss B. Novel vaccinia vector derived from the host range restricted and highly attenuated MVA strain of vaccinia virus. Dev Biol Stand. 1995; 84:195-200.
49. Martin R M, Brady J L, Lew A M. The need for IgG2c specific antiserum when isotyping antibodies from C57BL/6 and NOD mice. J Immunol Methods. 1998; 212(2): 187-92.
50. Liew F Y. TH1 and TH2 cells: a historical perspective. Nature Reviews Immunology. 2002; 2(1):55-60.
51. Mosmann T R, Cherwinski H, Bond M W, Giedlin M A, Coffman R L. Two types of murine helper T cell clone. I. Definition according to profiles of lymphokine activities and secreted proteins. J Immunol. 1986; 136(7):2348-57.
52. Seder R A, Paul W E. Acquisition of lymphokine-producing phenotype by CD4+ T cells. Annu Rev Immunol. 1994; 12:635-73.
53. Roberts A D, Woodland D L. Cutting edge: effector memory CD8+ T cells play a prominent role in recall responses to secondary viral infection in the lung. J Immunol. 2004; 172(11):6533-7.
54. Sia S F, Yan L M, Chin A W H, Fung K, Choy K T, Wong A Y L, et al. Pathogenesis and transmission of SARS-CoV-2 in golden hamsters. Nature. 2020; 583(7818):834-8.
55. Tostanoski L H, Wegmann F, Martinot A J, Loos C, McMahan K, Mercado N B, et al. Ad26 vaccine protects against SARS-CoV-2 severe clinical disease in hamsters. Nat Med. 2020; 26(11):1694-700.
56. Imai M, Iwatsuki-Horimoto K, Hata M, Loeber S, Halfmann P J, Nakajima N, et al. Syrian hamsters as a small animal model for SARS-CoV-2 infection and countermeasure development. Proc Natl Acad Sci USA. 2020; 117(28):16587-95.
57. Yahalom-Ronen Y, Tamir H, Melamed S, Politi B, Shifman O, Achdout H, et al. A single dose of recombinant VSV-G-spike vaccine provides protection against SARS-CoV-2 challenge. Nat Commun. 2020; 11(1):6402.
58. Rosenke K, Meade-White K, Letko M, Clancy C, Hansen F, Liu Y N, et al. Defining the Syrian hamster as a highly susceptible preclinical model for SARS-CoV-2 infection. Emerg Microbes Infec. 2020; 9(1):2673-84.
59. Baum A, Ajithdoss D, Copin R, Zhou A, Lanza K, Negron N, et al. REGN-COV2 antibodies prevent and treat SARS-CoV-2 infection in rhesus macaques and hamsters. Science. 2020; 370(6520):1110-5.
60. Song Z, Bao L, Yu P, Qi F, Gong S, Wang J, et al. SARS-CoV-2 Causes a Systemically Multiple Organs Damages and Dissemination in Hamsters. Front Microbiol. 2020; 11:618891.
61. Urata S, Maruyama J, Kishimoto-Urata M, Sattler R A, Cook R, Lin N, et al. Regeneration Profiles of Olfactory Epithelium after SARS-CoV-2 Infection in Golden Syrian Hamsters. ACS Chem Neurosci. 2021; 12(4):589-95.
62. Driouich J S, Cochin M, Lingas G, Moureau G, Touret F, Petit P R, et al. Favipiravir antiviral efficacy against SARS-CoV-2 in a hamster model. Nat Commun. 2021; 12(1): 1735.
63. Brocato R L, Kwilas S A, Kim R K, Zeng X, Principe L M, Smith J M, et al. Protective efficacy of a SARS-CoV-2 DNA vaccine in wild-type and immunosuppressed Syrian hamsters. NPJ Vaccines. 2021; 6(1):16.
64. Mohandas S, Yadav P D, Shete-Aich A, Abraham P, Vadrevu K M, Sapkal G, et al. Immunogenicity and protective efficacy of BBV152, whole virion inactivated SARS-CoV-2 vaccine candidates in the Syrian hamster model. iScience. 2021; 24(2):102054.
65. Munoz-Fontela C, Dowling W E, Funnell S G P, Gsell P S, Riveros-Balta A X, Albrecht R A, et al. Animal models for COVID-19. Nature. 2020; 586(7830):509-15.
66. Imai M, Iwatsuki-Horimoto K, Haifa M, Loeber S, Halfmann P J, Nakajima N, et al. Syrian hamsters as a small animal model for SARS-CoV-2 infection and countermeasure development. P Natl Acad Sci USA. 2020; 117(28):16587-95.
67. Chan J F W, Zhang A N J X, Yuan S F, Poon V K M, Chan C C S, Lee A C Y, et al. Simulation of the Clinical and Pathological Manifestations of Coronavirus Disease 2019 (COVID-19) in a Golden Syrian Hamster Model: Implications for Disease Pathogenesis and Transmissibility. Clin Infect Dis. 2020; 71(9):2428-46.
68. Li C, Chen Y X, Liu F F, Lee A C, Zhao Y, Ye Z H, et al. Absence of vaccine-enhanced disease with unexpected positive protection against SARS-CoV-2 by inactivated vaccine given within three days of virus challenge in Syrian hamster model. Clin Infect Dis. 2021.
69. Zarling J M, Eichberg J W, Moran P A, McClure J, Sridhar P, Hu S L. Proliferative and cytotoxic T cells to AIDS virus glycoproteins in chimpanzees immunized with a recombinant vaccinia virus expressing AIDS virus envelope glycoproteins. J Immunol. 1987; 139(4):988-90.
70. Hu S L, Zarling J M, Chinn J, Travis B M, Moran P A, Sias J, et al. Protection of macaques against simian AIDS by immunization with a recombinant vaccinia virus expressing the envelope glycoproteins of simian type D retrovirus. Proc Natl Acad Sci USA. 1989; 86(18):7213-7.
71. Hu S L, Plowman G D, Sridhar P, Stevenson U S, Brown J P, Estin C D. Characterization of a recombinant vaccinia virus expressing human melanoma-associated antigen p97. J Virol. 1988; 62(1):176-80.
72. Hu S L, Travis B M, Garrigues J, Zarling J M, Sridhar P, Dykers T, et al. Processing, Assembly, and Immunogenicity of Human-Immunodeficiency-Virus Core Antigens Expressed by Recombinant Vaccinia Virus. Virology. 1990; 179(1):321-9.
73. Wyatt L S, Earl P L, Moss B. Generation of Recombinant Vaccinia Viruses. Curr Protoc Microbiol. 2015; 39:14A41-A48.
74. Cotter C A, Earl P L, Wyatt L S, Moss B. Preparation of Cell Cultures and Vaccinia Virus Stocks. Curr Protoc Microbiol. 2015; 39:14A31-A38.
75. Chakrabarti S, Brechling K, Moss B. Vaccinia virus expression vector: coexpression of beta-galactosidase provides visual screening of recombinant virus plaques. Mol Cell Biol. 1985; 5(12):3403-9.
76. Kasani S K, Cheng H Y, Yeh K E, Chang S J, Hsu P W, Tung S Y, et al. Reply to “Bioinformatics Analysis of Differential Innate Immune Signaling in Macrophages by Wild-Type Vaccinia Mature Virus and a Mutant Virus with a Deletion of the A26 Protein”. J Virol. 2017; 91(24).
77. Izmailyan R, Hsao J C, Chung C S, Chen C H, Hsu P W, Liao C L, et al. Integrin betal mediates vaccinia virus entry through activation of PI3K/Akt signaling. J Virol. 2012; 86(12):6677-87.
78. Golde W T, Gollobin P, Rodriguez L L. A rapid, simple, and humane method for submandibular bleeding of mice using a lancet. Lab Animal. 2005; 34(9):39-43.
79. Rodrigues M V, de Castro S O, de Albuquerque C Z, Mattaraia V G D, Santoro M L. The gingival vein as a minimally traumatic site for multiple blood sampling in guinea pigs and hamsters. Plos One. 2017; 12(5).
80. Kuo T Y, Lin M Y, Coffman R L, Campbell J D, Traquina P, Lin Y J, et al. Development of CpG-adjuvanted stable prefusion SARS-CoV-2 spike antigen as a subunit vaccine against COVID-19. Sci Rep. 2020; 10(1):20085.
81. Ramakrishnan M A. Determination of 50% endpoint titer using a simple formula. World J Virol. 2016; 5(2):85-6.
82. Feng L, Wang Q, Shan C, Yang C, Feng Y, Wu J, et al. An adenovirus-vectored COVID-19 vaccine confers protection from SARS-COV-2 challenge in rhesus macaques. Nat Commun. 2020; 11(1):4207.
83. Haun B K, Lai C Y, Williams C A, Wong T A S, Lieberman M M, Pessaint L, et al. CoVaccine HT Adjuvant Potentiates Robust Immune Responses to Recombinant SARS-CoV-2 Spike S1 Immunization. Front Immunol. 2020; 11:599587.
84. Corman V M, Landt O, Kaiser M, Molenkamp R, Meijer A, Chu D K, et al. Detection of 2019 novel coronavirus (2019-nCoV) by real-time RT-PCR. Euro Surveill. 2020; 25(3).

What is claimed is:

1. A recombinant poxviral vector which comprises a polynucleotide encoding a SARS-CoV-2 spike protein in a poxviral vector for use in vaccinating a subject against SARS-CoV-2.

2. The recombinant poxviral vector of claim 1, wherein the poxviral vector is an orthopox viral vector.

3. The recombinant poxviral vector of claim 2, wherein the orthopox viral vector is selected from the group consisting of a camelpox viral vector, a cowpox viral vector, a monkey pox viral vector, a smallpox viral vector and a vaccinia vial vector.

4. The recombinant poxviral vector of claim 3, wherein the vaccinia vial vector is modified vaccine Ankara (MVA) or v-NY.

5. The recombinant poxviral vector of claim 1, wherein the recombinant poxviral vector lacks a functional thymidine kinase gene.

6. The recombinant poxviral vector of claim 1, wherein the polynucleotide is operatively-linked to a promoter.

7. The recombinant poxviral vector of claim 6, wherein the promoter is a poxviral promoter.

8. The recombinant poxviral vector of claim 7, wherein the promoter is a vaccinia viral early and late dual promoter.

9. The recombinant poxviral vector of claim 1, wherein the SARS-CoV-2 spike protein comprises the amino acid sequence of SEQ ID NO: 1, 2, 3, 4 or 5, or a functional variant thereof.

10. The recombinant poxviral vector of claim 1, which is v-NY-S (deposit accession number BCRC970077 or CNCM 1-5857).

11. An immunogenic composition against SARS-CoV-2 which comprises an effective amount of a recombinant poxviral vector and a physiologically acceptable vehicle, wherein the recombinant poxviral vector comprises a polynucleotide encoding a SARS-CoV-2 spike protein in a poxviral vector.

12. The immunogenic composition of claim 11, which further comprises an adjuvant.

13. The immunogenic composition of claim 11, wherein the recombinant poxviral vector is v-NY-S (deposit accession number BCRC970077 or CNCM I-5857).

14. A method for vaccinating a subject against SARS-CoV-2, comprising administering to the subject an effective amount of a recombinant poxviral vector or an immunogenic composition comprising the recombinant poxviral vector, wherein the recombinant poxviral vector comprises a polynucleotide encoding a SARS-CoV-2 spike protein in a poxviral vector.

15. The method of claim 14, wherein the recombinant poxviral vector or the immunogenic composition is administered via a route selected from the group consisting of intramuscular injection, subcutaneous injection, intranasal administration, intradermal injection, skin scarification and oral administration and any combination thereof.

16. The method of claim 14, wherein the recombinant poxviral vector or the immunogenic composition is administered to the subject once or more than once.

17. The method of claim 14, comprising a first administration, followed by a second administration, of the recombinant poxviral vector or the immunogenic composition.

18. The method of claim 17, wherein the first administration and the second administration are intramuscular injection.

19. The method of claim 17, wherein the first administration is skin scarification and the second administration is intramuscular injection.

20. The method of claim 17, wherein the second administration is about four weeks after the first administration.

21. The method of any of claims 17, wherein the same dose is given in the first administration and the second administration.

22. The method of any of claims 17, wherein a higher dose is given in the first administration than in the second administration.

23. The method of claim 14, wherein the recombinant poxviral vector comprises v-NY-S and/or MVA-S.

24. The method of claim 23, comprising administering an effective amount of v-NY-S first and administering an effective amount of MVA-S later to the subject.

25. The method of claim 24, wherein v-NY-S is administered via skin scarification and MVA-S is administered via intramuscular injection.

26. The method of claim 23, comprising administering a first amount of v-NY-S first via skin scarification and administering a second amount of MVA-S via intramuscular injection after at least four weeks of the administration of v-NY-S to the subject, wherein the second amount is higher than the first amount.

27. The method of claim 14, wherein the method is effective in inducing neutralizing antibodies and specific T_{H}1-biased immune responses and effector memory CD8+ T cells ragainst SARS-CoV-2 in the subject.

28. The method of claim 14, wherein the method is effective in reducing a disease or condition caused by SARS-CoV-2 infection in the subject.

29. The method of claim 28, wherein the disease or condition includes damages in organs or tissues in the subject, selected from the group consisting of lung, gastrointestinal tract, heart, kidney, liver, adrenal glands and/or testis.

30. The method of claim 28, wherein the disease or condition includes a pathological condition in lung, selected from the group consisting of diffuse congestion, shrinking of alveoli, hemorrhaging, immune cell infiltration and any combination thereof.


SEQUENCE LISTING (8 sequences)

SEQ ID NO: 1 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1270 aa, Severe acute respiratory syndrome coronavirus 2; identical to SEQ ID NO: 1

SEQ ID NO: 3 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1273 aa, Severe acute respiratory syndrome coronavirus 2
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1271 aa, Severe acute respiratory syndrome coronavirus 2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - DNA, 26 bp, synthetic construct
  source 1..26: mol_type = other DNA
acaggtacgttaatagttaatagcgt

SEQ ID NO: 7 - DNA, 22 bp, synthetic construct
  source 1..22: mol_type = other DNA
atattgcagcagtacgcacaca

SEQ ID NO: 8 - DNA, 26 bp, synthetic construct
  source 1..26: mol_type = other DNA
acactagccatccttactgcgcttcg